(19) United States
(12) Patent Application Publication
(10) Pub. No.: US 2016/0202273 A1
(43) Pub. Date: Jul. 14, 2016

(54) BIOMARKERS ASSOCIATED WITH DIABETES AND FIBROSIS

(71) Applicant: CAMBRIDGE UNIVERSITY HOSPITALS NHS FOUNDATION TRUST, Cambridge, Cambridgeshire (GB)

(72) Inventors: Graeme J.M. Alexander, Cambridge (GB); Aloysius Dominic Aravinthan, London (GB)

(21) Appl. No.: 14/916,761

(22) PCT Filed: Sep. 4, 2014
(86) PCT No.: PCT/GB2014/052688

§ 371 Date: Mar. 4, 2016

(30) Foreign Application Priority Data
Sep. 4, 2013 (GB) 1315747.4

(51) Int. Cl. G01N 33/68; H01J 49/00; C12Q 1/68

(52) U.S. Cl. CPC G01N 33/6893; C12Q 1/6883; H01J 49/0027; C12Q 2600/118; G01N 2800/52; G01N 2800/50; G01N 2800/085; G01N 2800/042

(57) ABSTRACT

The present invention provides one or more biomarkers that are associated with fibrosis, e.g. liver fibrosis, and/or diabetes or a prediabetic condition. The invention further provides methods for diagnosing, prognosing and monitoring fibrosis, as well as methods for diagnosing, prognosing and monitoring diabetes, or a prediabetic condition, using said one or more biomarkers, in addition to kits for carrying out such methods.

The present invention relates to one or more biomarkers associated with fibrosis and diabetes or a prediabetic condition. More particularly, the invention relates to methods for diagnosing, prognosing and monitoring fibrosis, as well as to methods for diagnosing, prognosing and monitoring diabetes or a prediabetic condition using said one or more biomarkers.

Fibrosis is the formation of excessive fibrous tissue. Fibrosis may be the result of a response to necrosis, injury, or chronic inflammation, which may be induced by a wide variety of agents. Fibrosis is most common in the liver, heart, lung, peritoneum, and kidney.

Determination of the extent of fibrosis is important in clinical practice for the prognosis and management of many diseases. For example, the extent of hepatic fibrosis reflects disease severity and is vital for determining chronic liver disease therapy. Liver biopsy is currently the “gold standard” for grading and staging liver disease. However, liver biopsy is an invasive procedure, which not only incurs substantial risk of complications, but also may result in considerable error in disease staging due to variability in histological interpretation.

Diabetes is a serious illness characterized by a loss of the ability to regulate blood glucose levels. Patients with Type 1 Diabetes exhibit little or no insulin secretion as manifested by low or undetectable levels of insulin. Type 2 Diabetes results from insensitivity to insulin, and accounts for 90% of diabetes worldwide. Risk factors for Type 2 Diabetes include high cholesterol, high blood pressure, ethnicity, and genetic factors, such as a family history of diabetes. The majority of patients with Type 2 Diabetes are obese, and obesity itself may cause or aggravate insulin resistance.

It is well documented that the prediabetic state can be present for ten or more years before detection; yet few prediabetics are identified and treated. A major reason is that no simple laboratory test exists to determine the actual risk of an individual to develop diabetes.

Accordingly, there is a need in the art for a convenient method for screening persons at risk for developing fibrosis or diabetes or for identifying patients who have developed these diseases. This need is addressed by the present invention, which provides methods for diagnosing, prognosing and monitoring the progression of fibrosis or diabetes or a prediabetic condition.

The present invention is premised on the discovery of biomarkers that can be identified before overt disease is apparent. The biomarkers can be used as early diagnostic tools, as well as prognostic indicators. The biomarkers may also be used to monitor the disease.

Thus, in one aspect, the present invention provides a method for diagnosing, prognosing or evaluating the risk of fibrosis, e.g. liver fibrosis, in a patient, comprising determining an effective amount of one or more biomarkers in a sample obtained from a patient, wherein the one or more biomarkers are selected from:
(a) a protein listed in Table 1 or Table 2 or a fragment, variant or derivative thereof;
(b) a protein expressed from a gene listed in Table 1 or Table 2 or a fragment, variant or derivative thereof; or
(c) a nucleic acid expressed from a gene listed in Table 1 or Table 2 or a fragment thereof.

In one embodiment, the one or more biomarkers are selected from the group consisting of CSF1, LYVE1, MRGPRX4 and IL-18 (as defined in Tables 1 and 2). In another embodiment, the one or more biomarkers are selected from the group consisting of G6PC, MVP, PGM2L1, ANXA3, SERPINB8, MYOF and SPARC (as defined in Tables 1 and 2).

In one embodiment, the method for diagnosing, prognosing or evaluating the risk of fibrosis comprises:
(i) determining an effective amount of the one or more biomarkers in a sample obtained from a patient; and
(ii) comparing the effective amount to a reference value.

In one embodiment, an increase in the effective amount relative to the reference value indicates that the patient has, or may be at risk of developing, fibrosis. The increase can be, for example, at least 5%, at least 10%, at least 15%, at least 20%, at least 25%, at least 30%, at least 35%, at least 40%, at least 45%, or at least 50% of the reference value. The increase in the effective amount of the one or more biomarkers is preferably statistically significant. By “statistically significant”, it is meant that the alteration is greater than what might be expected to happen by chance alone. Statistical significance can be determined by any method known in the art.

In one embodiment, a decrease in the effective amount relative to the reference value indicates that the patient has, or may be at risk of developing, fibrosis. The decrease can be, for example, at least 5%, at least 10%, at least 15%, at least 20%, at least 25%, at least 30%, at least 35%, at least 40%, at least 45%, or at least 50% of the reference value. The decrease in the effective amount of the one or more biomarkers is preferably statistically significant. By “statistically significant”, it is meant that the alteration is greater than what might be expected to happen by chance alone. Statistical significance can be determined by any method known in the art.

In one embodiment, the sample for use in the method for diagnosing, prognosing or evaluating the risk of fibrosis is selected from the group consisting of blood, saliva, tears, sputum, urine, cerebral spinal fluid, cells, a cellular extract, a tissue specimen, a tissue biopsy, a stool specimen, or any combination thereof. In one embodiment, the sample is a blood sample. In a preferred embodiment, the sample is blood plasma.

Accordingly, the present invention allows the skilled person to diagnose subjects who have developed fibrosis, e.g. liver fibrosis, including those subjects who are asymptomatic for fibrosis, and also to determine the severity of fibrosis, by detection of the one or more biomarkers. Thus, the present invention enables the skilled person to make a definitive or near definitive determination as to whether a subject is presently affected by fibrosis.

In addition, detection of the one or more biomarkers of the invention allows the skilled person to determine whether a subject has an increased likelihood of having or developing fibrosis, e.g. liver fibrosis, when compared to a control subject or to the general population. Thus, the present invention enables one of skill in the art to identify or otherwise assess those subjects who do not exhibit any symptoms of fibrosis, but who nonetheless may be at risk for developing fibrosis.

Furthermore, the one or more biomarkers may be used in the prognosis of fibrosis, e.g. liver fibrosis. Thus, the present invention may be used in the act of foretelling the course of fibrosis, including e.g. the prospect of morbidity, mortality and the development of disease-related complications.

In another aspect, the present invention provides a method for diagnosing, prognosing or evaluating the risk of diabetes or a prediabetic condition in a patient, comprising determining an effective amount of one or more biomarkers in a sample obtained from a patient, wherein the one or more biomarkers are selected from:
(a) a protein listed in Table 1 or Table 2 or a fragment, variant or derivative thereof;
(b) a protein expressed from a gene listed in Table 1 or Table 2 or a fragment, variant or derivative thereof; or
(c) a nucleic acid expressed from a gene listed in Table 1 or Table 2 or a fragment thereof.

In one embodiment, the one or more biomarkers are selected from the group consisting of CSF1, MRGPRX4, IL-18, CYR61 and LYVE1 (as defined in Tables 1 and 2). In another embodiment, the one or more biomarkers are selected from the group consisting of G6PC, MVP, PGM2L1, ANXA3, SERPINB8 and MYOF (as defined in Tables 1 and 2).

In one embodiment, the method for diagnosing, prognosing or evaluating the risk of diabetes or a prediabetic condition comprises:
(i) determining an effective amount of one or more biomarkers in a sample obtained from a patient; and
(ii) comparing the effective amount to a reference value.

In one embodiment, an increase in the effective amount relative to the reference value indicates that the patient has, or may be at risk of developing, diabetes or a prediabetic condition. The increase can be, for example, at least 5%, at least 10%, at least 15%, at least 20%, at least 25%, at least 30%, at least 35%, at least 40%, at least 45%, or at least 50% of the reference value. The increase in the effective amount of the one or more biomarkers is preferably statistically significant. By “statistically significant”, it is meant that the alteration is greater than what might be expected to happen by chance alone. Statistical significance can be determined by any method known in the art.

In one embodiment, a decrease in the effective amount relative to the reference value indicates that the patient has, or may be at risk of developing, diabetes or a prediabetic condition. The decrease can be, for example, at least 5%, at least 10%, at least 15%, at least 20%, at least 25%, at least 30%, at least 35%, at least 40%, at least 45%, or at least 50% of the reference value. The decrease in the effective amount of the one or more biomarkers is preferably statistically significant. By “statistically significant”, it is meant that the alteration is greater than what might be expected to happen by chance alone. Statistical significance can be determined by any method known in the art.

In one embodiment, the sample for use in the method for diagnosing, prognosing or evaluating the risk of diabetes or a prediabetic condition is selected from the group consisting of blood, saliva, tears, sputum, urine, cerebral spinal fluid, cells, a cellular extract, a tissue specimen, a tissue biopsy, a stool specimen, or any combination thereof. In one embodiment, the sample is a blood sample. In a preferred embodiment the sample is blood plasma.

Accordingly, the present invention allows the skilled person to diagnose subjects who have developed diabetes or a prediabetic condition, including those subjects who are asymptomatic for diabetes or a prediabetic condition, by detection of the one or more biomarkers. Thus, the present invention enables the skilled person to make a definitive or near definitive determination as to whether a subject is presently affected by diabetes or a prediabetic condition.

In addition, detection of the one or more biomarkers allows the skilled person to determine whether a subject has an increased likelihood of having or developing diabetes or a prediabetic condition when compared to a control subject or to the general population. Thus, the present invention enables one of skill in the art to identify or otherwise assess those subjects who do not exhibit any symptoms of diabetes or a prediabetic condition, but who nonetheless may be at risk for developing diabetes or a prediabetic condition.

Furthermore, the one or more biomarkers may be used in the prognosis of diabetes or a prediabetic condition. Thus, the present invention may be used in the act of foretelling the course of diabetes or a prediabetic condition, including e.g. the prospect of morbidity, mortality and the development of disease-related complications.

Using the biomarkers of the present invention, both fibrosis, e.g. liver fibrosis, and diabetes or a prediabetic condition can be diagnosed at an early stage, thus enabling clinicians to administer early therapeutic interventions, improving the likelihood of a successful therapeutic outcome. In some embodiments, the methods of the present invention are practiced in conjunction with other methods known to the skilled person which provide a means of diagnosing, prognosing, or determining the risk of fibrosis and diabetes or a prediabetic condition.

In a further aspect, the present invention provides a method for monitoring fibrosis, e.g. liver fibrosis, comprising:
(i) determining an effective amount of one or more biomarkers in a first sample obtained from a patient at a first time period;
(ii) determining an effective amount of the one or more biomarkers in a sample obtained from the patient at one or more later time periods; and
(iii) comparing the effective amount determined in step (ii) to the effective amount detected in step (i) to determine a difference in the effective amount of the one or more biomarkers,
   wherein the one or more biomarkers are selected from:
(a) a protein listed in Table 1 or Table 2 or a fragment, variant or derivative thereof;
(b) a protein expressed from a gene listed in Table 1 or Table 2 or a fragment, variant or derivative thereof; or
(c) a nucleic acid expressed from a gene listed in Table 1 or Table 2 or a fragment thereof.

In one embodiment, the one or more biomarkers are selected from the group consisting of CSF1, LYVE1, MRGPRX4 and IL-18 (as defined in Tables 1 and 2). In another embodiment, the one or more biomarkers are selected from the group consisting of G6PC, MVP, PGM2L1, ANXA3, SERPINB8, MYOF and SPARC (as defined in Tables 1 and 2).

In a further aspect, the present invention provides a method for monitoring diabetes or a prediabetic condition, comprising:
(i) determining an effective amount of one or more biomarkers in a first sample obtained from a patient at a first time period;
(ii) determining an effective amount of the one or more biomarkers in a sample obtained from the patient at one or more later time periods; and
(iii) comparing the effective amount determined in step (ii) to the effective amount detected in step (i) to determine a difference in the effective amount of the one or more biomarkers,
   wherein the one or more biomarkers are selected from:
(a) a protein listed in Table 1 or Table 2 or a fragment, variant or derivative thereof;
(b) a protein expressed from a gene listed in Table 1 or Table 2 or a fragment, variant or derivative thereof; or
(c) a nucleic acid expressed from a gene listed in Table 1 or Table 2 or a fragment thereof.

In one embodiment, the one or more biomarkers are selected from the group consisting of CSF1, MRGPRX4, IL-18, CYR61 and LYVE1 (as defined in Tables 1 and 2). In another embodiment, the one or more biomarkers are selected from the group consisting of G6PC, MVP, PGM2L1, ANXA3, SERPINB8 and MYOF (as defined in Tables 1 and 2).

Thus, the monitoring methods of the present invention may (a) provide an indication as to disease severity, (b) aid determination as to the correct course of treatment, (c) permit evaluation of response to treatment, (d) permit determination as to whether to continue or cease treatment, (e) provide a means of disease staging or (f) permit determination as to clinical outcome.

In a related aspect, the method of the present invention monitors the effectiveness of a treatment regimen for fibrosis, e.g. liver fibrosis, or diabetes or a prediabetic condition. Thus, the method may comprise:
(i) determining an effective amount of one or more biomarkers in a first sample obtained from a patient at a first time period before or during the course of treatment;
(ii) determining an effective amount of the one or more biomarkers in a sample obtained from the patient at one or more later time periods during or after treatment; and
(iii) comparing the effective amount determined in step (ii) to the effective amount detected in step (i) to determine a difference in the effective amount of the one or more biomarkers,
   wherein the one or more biomarkers are selected from:
(a) a protein listed in Table 1 or Table 2 or a fragment, variant or derivative thereof;
(b) a protein expressed from a gene listed in Table 1 or Table 2 or a fragment, variant or derivative thereof; or
(c) a nucleic acid expressed from a gene listed in Table 1 or Table 2 or a fragment thereof.

A difference in the effective amount of the one or more biomarkers measured by the monitoring methods of the present invention can comprise an increase or a decrease in the effective amount of the one or more biomarkers. The increase or decrease in the “effective amount” of a biomarker can be, for example, at least 5%, at least 10%, at least 15%, at least 20%, at least 25%, at least 30%, at least 35%, at least 40%, at least 45%, or at least 50% of the reference value. The difference in the effective amount of the biomarker is preferably statistically significant. By “statistically significant”, it is meant that the alteration is greater than what might be expected to happen by chance alone.

The increase or decrease in the effective amount can indicate progression or development of the disease, or the lack of efficacy of one or more treatment regimens. Alternatively, the increase or decrease in the effective amount can indicate regression of the disease, or can be indicative of the success of one or more treatment regimens. The term “progression” refers to an increase in the symptoms of a disease or disorder. Similarly, the term “regression” refers to a decrease in the symptoms of disease or disorder.

In one embodiment, the sample for use in the monitoring methods of the present invention is selected from the group consisting of blood, saliva, tears, sputum, urine, cerebral spinal fluid, cells, a cellular extract, a tissue specimen, a tissue biopsy, a stool specimen, or any combination thereof. In one embodiment the sample is a blood sample. In a preferred embodiment, the sample is blood plasma.

In another aspect, the invention provides a test kit for carrying out the methods of the invention. The kit may comprise reagents for measuring the one or more biomarkers of the invention. The kit may further comprise control formulations (positive and/or negative), and/or a detectable label. Instructions for carrying out the assay may also be included in the kit.

In one embodiment, the reagents comprise nucleic acids that can specifically identify one or more nucleic acid biomarkers of the invention by having nucleic acid sequences complementary to a portion of the nucleic acid biomarkers. The nucleic acid sequences can be 1000, 500, 200, 150, 100, 50, 25, 10 nucleotides or fewer in length.

In another embodiment, the reagents comprise a plurality of antibodies that specifically bind one or more of the protein biomarkers of the invention. In accordance with this embodiment, the kit may include antibodies, fragments or derivatives thereof (e.g., Fab, F(ab′)2, Fv, or scFv fragments) that are specific for the biomarkers of the present invention. In one embodiment, the antibodies may be detectably labelled. The antibodies may be either monoclonal or polyclonal antibodies.

### DETAILED DESCRIPTION

Without being bound by any particular theory, it is believed that there is a direct link between hepatocyte senescence and both the stage of fibrosis and progression of fibrosis in patients with chronic liver disease. Senescent hepatocytes were found to secrete factors that were able to up-regulate genes encoding signalling factors capable of promoting the proliferation and activation of hepatic stellate cells (the major cell type involved in fibrosis). Furthermore, a ‘geographic association’ was found between senescent hepatocytes and activated hepatic stellate cells in patients with chronic liver disease. These findings suggest that senescent hepatocytes are responsible for driving the development and progression of fibrosis in chronic liver disease. Indeed, a strong significant relationship was found to exist between the proportion of senescent hepatocytes and the later likelihood of developing an adverse liver-related outcome in this group of patients including death.

Further investigation by the present inventors also revealed that senescent hepatocytes exhibit altered expression of several genes involved in the metabolism of glucose and essential signal transduction pathways. Notably, the present inventors found that two of the four downstream effector pathways in the PI3K-Akt cascade are defective, indicating selective insulin resistance in senescent hepatocytes. Although insulin resistance is common in patients with chronic liver disease (as many as 70% of patients with cirrhosis demonstrate impaired glucose tolerance), to date, the role of the liver and more specifically hepatocytes, in related insulin resistance complicating chronic liver disease has never been elucidated. Accordingly, the discovery by the present inventors of a role for senescence in the development of insulin resistance complicating chronic liver disease is significant.

In conclusion, the present inventors believe that hepatocyte senescence, progressive fibrosis and insulin resistance complicating chronic liver disease are inter-related. Based on their findings, the present inventors have identified biomarkers that are capable of distinguishing between healthy individuals and those with liver fibrosis and/or diabetes mellitus.

### The Patient

The terms “individual”, “subject”, and “patient”, are used interchangeably herein to refer to a mammalian subject for whom diagnosis, prognosis, treatment, therapy or disease monitoring is desired. The mammal can be a human, non-human primate, mouse, rat, dog, cat, horse or cow, but is not limited to these examples. In one preferred embodiment, the individual, subject, or patient is a human, e.g. a male or female.

In the methods of the present invention, the subject may not have been previously diagnosed as having the disease (i.e. diabetes or fibrosis). The subject may also be one who has been previously diagnosed as having the disease (i.e. diabetes or fibrosis). Alternatively, the subject may be one who does not exhibit disease risk factors or one who is asymptomatic for the disease (i.e. diabetes or fibrosis). A subject can also be one who is suffering from or at risk of developing the disease.

### The Sample

The sample for use in the methods of the present invention can encompass liquid samples of biological origin, solid tissue samples such as a biopsy specimen, or tissue cultures or cells derived therefrom and the progeny thereof. The precise sample to be taken from an individual may vary, but is typically minimally invasive and is easily performed by conventional techniques known in the art.

In one embodiment, the sample for use in the methods of the present invention is selected from the group consisting of blood, saliva, tears, sputum, urine, cerebral spinal fluid, cells, a cellular extract, a tissue specimen, a tissue biopsy, or a stool specimen. Furthermore, pools or mixtures of the above-mentioned samples may be employed.

In one embodiment, the sample is derived from urine. In another embodiment, the sample is derived from blood. The term blood comprises whole blood, blood serum (henceforth “serum”) and blood plasma (henceforth “plasma”). Serum and plasma are derived from blood and thus may be considered as specific subtypes within the broader genus “blood”. In a preferred embodiment, the sample is plasma. Processes for obtaining serum or plasma from blood are known in the art. For example, it is known in the art that blood can be subjected to centrifugation in order to separate red blood cells, white blood cells, and plasma. Serum is defined as plasma that lacks clotting factors. Serum can be obtained by centrifugation of blood in which the clotting process has been triggered. Optionally, this can be carried out in specialised centrifuge tubes designed for this purpose.

The methods of the present invention may utilise samples that have undergone minimal or zero processing before testing. They may also utilise samples that have been manipulated, in any way, after procurement, such as treatment with reagents, solubilisation, or enrichment for certain components.

The sample for use in the methods of the present invention can be derived from either blood or urine that has undergone processing after being obtained from a test subject. Alternatively, a sample can be derived from blood or urine that has not undergone any processing after being obtained from a test subject. By way of example, a urine sample obtained from a test subject may be tested directly using the method of the present invention, without further processing. Serum and plasma samples can be readily obtained from blood samples using simple and readily available techniques that are well known in the art, as described above.

The methods of the present invention are in vitro methods. Thus, the methods of the present invention can be carried out in vitro on an isolated sampled that has been obtained from a subject.

### Determining the “Effective Amount” of One or More Biomarkers

Determining the “effective amount” of one or more biomarkers in a sample means quantifying the biomarker by determining, for example, the relative or absolute amount of the biomarker. It will be appreciated that the assay methods do not necessarily require measurement of absolute values of biomarker, unless it is desired, because relative values are sufficient for many applications of the invention. Accordingly, the “effective amount” can be the (absolute) total amount of the biomarker that is detected in a sample, or it can be a “relative” amount, e.g., the difference between the biomarker detected in a sample and e.g. another constituent of the sample. In some embodiments, the effective amount of the biomarker may be expressed by its concentration in a sample, or by the concentration of an antibody that binds to the biomarker.

When the biomarker is a protein as described herein (e.g., a protein listed in Table 1 or Table 2 or a protein expressed from a gene listed in Table 1 or Table 2), determining the “effective amount” of a biomarker can also encompass the analysis of the activity level (e.g. binding or enzymatic activity) of the protein.

In one embodiment, the methods of the present invention comprise determining the effective amount of a single biomarker of the present invention. An example of a single biomarker that may be measured is LYVE1 (as defined in Tables 1 or 2).

In another embodiment, the methods of the present invention comprise determining the effective amount of at least two (e.g. at least 2, 3, 4, 5, 6, 7, 8, 9, 10, 11, 12, 13, 14, 15, 16, 17, 18, 19, 20, 25, 30, 35, 40, 45, 50) biomarkers of the invention. By measuring multiple biomarkers, conclusions can be reached that are more precise and with higher confidence. The multiple biomarkers may be related by their function, for example, proteins associated with immune-mediated and inflammatory pathways, serum enzymes and structural proteins.

When the effective amounts of two or more biomarkers are determined, the methods of the present invention may determine the effective amount of each biomarker. Alternatively, the methods of the invention may determine the cumulative effective amount of all the biomarkers. Alternatively, the effective amount of the two or more biomarkers can be combined with each other in a formula to form an index value.

Measurement of the one or more biomarkers can be performed by any method that provides satisfactory analytical specificity, sensitivity and precision. The invention thus encompasses the use of those methods known to a person skilled in the art to measure the effective amount of one or more biomarkers for each of the purposes of diagnosing, prognosing, monitoring and determining the risk of fibrosis, e.g. liver fibrosis, and/or diabetes or a prediabetic condition in a subject.

The effective amount of the biomarker can be determined at the protein or nucleic acid level using any method known in the art. The particular preferred method for determining the effective amount of the one or more biomarkers will depend in part on the identity and nature of the biomarker.

For example, when the biomarker is a nucleic acid, the biomarker may be detected using any method well known to those skilled in the art. For example, nucleic acid levels can be determined using solution hybridization procedures as well as solid-phase hybridization procedures in which the probe or sample is immobilized on a solid support.

Other examples of useful methods include amplification methods such as target and signal amplification methods. These include PCR (polymerase chain reaction) and reverse-transcriptase-PCR (RT-PCR); transcription mediated amplification (Gen-Probe Incorporated; San Diego, Calif.); branched chain DNA (bDNA) amplification (Bayer Diagnostics; Emeryville, Calif.); strand displacement amplification (SDA; Becton Dickinson; Franklin Lakes, N.J.); and ligase chain reaction (LCR) amplification (Abbott Laboratories; Abbott Park, Ill.). Additional methods useful in the invention include RNase protection; Northern analysis or other RNA blot, dot blot or membrane-based technology; dip stick; pin; and two-dimensional array immobilized onto a chip. Conditions are well known in the art for quantitative determination of mRNA levels using both solution and solid phase hybridization procedures.

The polymerase chain reaction (PCR) RT-PCR can be useful in the methods of the invention. PCR or RT-PCR can be performed with isolated RNA or crude or partially fractionated samples, for example, cells pelleted from a whole blood sample. PCR methods are well known in the art.

When the biomarker is a protein, or a fragment, polymorphism, mutant, post-translationally modified, or otherwise modified, or processed form of such a protein, the biomarker may be detected using methods including, but not limited to: immunoassay methods (e.g. radioimmunoassay, immunoblotting, immunoprecipitation, immunofluorescence, enzyme-linked immunosorbant assay), separation-based methods (e.g., agarose gel electrophoresis, sodium dodecyl sulfate-polyacrylamide gel electrophoresis (SDS-PAGE), Tris-HCl polyacrylamide gels, non-denaturing protein gels, two-dimensional gel electrophoresis (2DE), and the like), or protein-based methods (e.g., surface enhanced laser desorption ionization (SELDI), matrix-assisted laser desorption ionization-time of flight (MALDI-TOF), high performance liquid chromatography (HPLC), liquid chromatography with or without mass spectrometry (LC/MS), tandem LC/MS, protein arrays, peptide arrays, and antibody assays).

Protein biomarkers can be typically detected by contacting a sample from the subject with an antibody which binds the protein biomarker. The antibody may be monoclonal, polyclonal, chimeric, or a fragment of the foregoing, and the step of detecting the reaction product may be carried out with any suitable immunoassay.

Microarray technology can be used as a tool for analysing gene or protein expression.

### Reference Value

The “reference value” refers to a value obtained from a control subject or population whose disease state is known. A reference value can be determined for any particular population, subpopulation, or group of subjects according to standard methods well known to those of skill in the art.

The reference value may be determined by quantifying the amount of a biomarker in a sample obtained from one or more normal subjects. As used herein, “normal” refers to a subject or group of subjects who are in a healthy state, e.g. patients who have not shown any symptoms of the disease, have not been diagnosed with the disease and/or are not likely to develop the disease. Preferably said normal subject(s) is not on medication affecting the disease and has not been diagnosed with any other disease. More preferably normal subjects have similar sex, age and body mass index (BMI) as compared with the test subject. Application of standard statistical methods used in medicine permits determination of normal levels of expression, as well as significant deviations from such normal levels.

Alternatively, the reference value may be determined by quantifying the amount of a biomarker in a sample obtained from one or more subjects suffering from the disease. More preferably such subjects have similar sex, age and body mass index (BMI) as compared with the test subject.

In one embodiment, the reference value is the effective amount of a biomarker in a sample or samples derived from a single subject. Alternatively, the reference value may be derived by pooling data obtained from two or more (e.g. at least three, four, five, 10, 15, 20 or 25) subjects and calculating an average (for example, mean or median) effective amount for a biomarker. Thus, the reference value may reflect the average amount of a biomarker in a given population of subjects. Said amounts may be expressed in absolute or relative terms, in the same manner as described above in relation to the sample that is to be tested using the method of the invention.

When comparing between the sample and the reference value, the way in which the amounts are expressed is matched between the sample and the reference value. Thus, an absolute amount can be compared with an absolute amount, and a relative amount can be compared with a relative amount.

The reference value may be derived from the same sample as the sample that is being tested, thus allowing for an appropriate comparison between the two. Thus, by way of example, if the sample is derived from urine, the reference value is also derived from urine. Alternatively, if the sample is a blood sample (e.g. a plasma or a serum sample), then the reference value will also be a blood sample (e.g. a plasma sample or a serum sample, as appropriate).

When the effective amounts of two or more biomarkers are determined, the method may comprise comparing the effective amount of each biomarker to its corresponding reference value. When the cumulative effective amount of all the biomarkers is determined, the method may comprise comparing the cumulative effective amount to a corresponding reference value. When the effective amount of the two or more biomarkers are combined with each other in a formula to form an index value, the index value can be compared to a corresponding reference index value derived in the same manner.

### Fibrosis

In one embodiment, the subject has, or is at risk of having fibrosis. Thus, the present invention relates to a method for diagnosing, prognosing, or evaluating the risk of fibrosis in a patient or to a method for monitoring the progression of fibrosis.

In one embodiment, the fibrosis affects an organ or tissue selected from the group consisting of, liver, prostate, breast, bladder, pancreas, lung, heart, nervous system, skin, kidney, bone marrow, lymph nodes, periureteric, endomyocardium, and retroperitoneum. Diseases associated with fibrosis in these organs and tissues include, but are not limited to, diffuse parenchymal lung disease, post-vasectomy pain syndrome, tuberculosis, sickle-cell anemia, rheumatoid arthritis, progressive massive fibrosis, idiopathic pulmonary fibrosis, renal fibrosis, myelofibrosis, cardiac fibrosis, pancreatic fibrosis, skin fibrosis, scleroderma, intestinal fibrosis or strictures, and mediastinal fibrosis. Fibrosis in all of these organs and tissues is characterized by the formation of excess fibrous connective tissue

In one embodiment, the fibrosis affects the liver. The methods of the invention can therefore be used for diagnosing/prognosing, monitoring the progression or evaluating the risk of liver fibrosis in an individual having, for example, viral hepatitis such as hepatitis A, B or C virus or a human immunodeficiency virus (HIV) such as HIV-1; chronic persistent hepatitis or chronic active hepatitis; autoimmune liver disease such as autoimmune hepatitis; alcohol-related liver disease; fatty liver disease, including non-alcoholic steatohepatitis (NASH); primary biliary cirrhosis; primary sclerosing cholangitis, biliary atresia; liver disease resulting from medical treatment (drug-induced liver disease); a metabolic disease such as alpha-1-antitrypsin deficiency or haemochromatosis; or a congenital liver disease.

The methods of the invention can also be used for prognosing and monitoring the progression in a patient of viral hepatitis such as hepatitis A, B or C virus or a human immunodeficiency virus (HIV) such as HIV-1; chronic persistent hepatitis or chronic active hepatitis; autoimmune liver disease such as autoimmune hepatitis; alcohol-related liver disease; fatty liver disease, including non-alcoholic steatohepatitis (NASH); primary biliary cirrhosis; primary sclerosing cholangitis, biliary atresia; liver disease resulting from medical treatment (drug-induced liver disease); a metabolic disease such as alpha-1-antitrypsin deficiency or haemochromatosis; or a congenital liver disease.

Periodic monitoring of liver fibrosis in individuals can be conveniently performed using the non-invasive methods of the invention, without the risks associated with liver biopsy.

### Diabetes or a Prediabetic Condition

In one embodiment, the subject has, or is at risk of having diabetes or a prediabetic condition. Thus, the present invention relates to a method for diagnosing, prognosing, or evaluating the risk of diabetes or a prediabetic condition in a patient or to a method for monitoring the progression of diabetes or a prediabetic condition.

In one embodiment, the term “diabetes” encompasses Type 2 Diabetes. The prediabetic condition encompasses a metabolic state that is intermediate between normal glucose homeostasis, metabolism, and states seen in Diabetes Mellitus. Examples of prediabetic conditions include, without limitation, Metabolic Syndrome (“Syndrome X”), Impaired Glucose Tolerance (IGT), Impaired Fasting Glycemia (IFG), insulin resistance or other Diabetes-related condition. “Insulin resistance” refers to a condition in which the cells of the body become resistant to the effects of insulin, that is, the normal response to a given amount of insulin is reduced. As a result, higher levels of insulin are needed in order for insulin to exert its effects. A prediabetic condition can also refer to those subjects who will, or are predicted to convert to frank Type 2 Diabetes within a given time period at a higher rate than that of the normal population.

### Treatment

In other embodiments, any of the aforementioned methods further include treating fibrosis, e.g. liver fibrosis, and/or one or more symptoms associated with fibrosis.

In one embodiment, the method includes, responsive to the effective amount of the one or more biomarkers in a sample obtained from a patient, administering to the patient a therapy for fibrosis.

In yet another aspect, the invention features a method of treating or preventing one or more symptoms associated with fibrosis, e.g. liver fibrosis in a subject. The method includes determining an effective amount of one or more biomarkers in a sample obtained from a patient that has, or is at risk of having, fibrosis, wherein the one or more biomarkers are selected from:
(a) a protein listed in Table 1 or Table 2 or a fragment, variant or derivative thereof;
(b) a protein expressed from a gene listed in Table 1 or Table 2 or a fragment, variant or derivative thereof; or
(c) a nucleic acid expressed from a gene listed in Table 1 or Table 2 or a fragment thereof
   and, responsive to said value, administering to the patient a therapy for fibrosis.

In certain embodiments, the therapy may include one or more of the following: the removal of the causative agent of fibrosis (e.g. the cessation of alcohol intake or a marked reduction in alcohol intake), organ transplantation, or the administration of therapeutic agents such as corticosteroids (e.g. prednisolone), antioxidants (e.g. vitamin E, silymarin, phosphatidycholine, S-adenosyl-L-methionine), growth factors (e.g. IGF, hepatocyte growth factor, cardiotrophone (including delivery by gene therapy)), inhibitors of signal transduction pathways involved in liver fibrogenesis (e.g. pentoxifylline (a phosphodiesterase inhibitor), amiloride (an Na⁺/H⁺ pump inhibitor) and S-farnesylthiosalicylic acid (a Ras antagonist)), ligands of PPARα and/or PPARγ (e.g. thiazolindiones), renin-angiotensin inhibitors, herbal compounds (e.g. sho-saiko-to, glycyrrhizin and savia miltiorhiza), inhibitors of collagen production (e.g. prolyl-4 hydroxylase and halofuginone), and immunosuppressants (e.g. rapamycin), among others.

In certain embodiments, the method of treatment includes one such therapy. In certain embodiments, the method of treatment includes a combination of two or more such therapies.

In other embodiments, any of the aforementioned methods further include treating diabetes or a prediabetic condition and/or one or more symptoms associated with diabetes or a prediabetic condition.

In one embodiment, the method includes, responsive to the effective amount of the one or more biomarkers in a sample obtained from a patient, administering to the patient a therapy for diabetes or a prediabetic condition.

In yet another aspect, the invention features a method of treating or preventing one or more symptoms associated with diabetes or a prediabetic condition in a subject. The method includes determining an effective amount of one or more biomarkers in a sample obtained from a patient that has, or is at risk of having, diabetes or a prediabetic condition, wherein the one or more biomarkers are selected from:
(a) a protein listed in Table 1 or Table 2 or a fragment, variant or derivative thereof;
(b) a protein expressed from a gene listed in Table 1 or Table 2 or a fragment, variant or derivative thereof; or
(c) a nucleic acid expressed from a gene listed in Table 1 or Table 2 or a fragment thereof
   and, responsive to said value, administering to the patient a therapy for diabetes or a prediabetic condition.

In certain embodiments, the therapy may include one or more of the following: dietary management, organ transplantation or the administration of therapeutic agents such as insulin secretagogues (e.g. sulfonylureas such as glyburide, gliclazide, glipizide and glimepiride or meglitinides such as repaglinide, nateflinde and mitiglinide), GLP-1 receptor agonists (e.g. exenatide, liraglutide, lixisenatide and albiglutide), sodium-glucose cotransporter-2-inhibitors (e.g. dapagliflozin, canagliglozin and empagliflozin), DPP-4 inhibitors (e.g. vildagliptin, anagliptin, teneligliptin, sitagliptin, saxagliptin, gemigliptin, alogliptin, and linagliptin), alpha-glucosidase inhibitors (e.g. acarbose, miglitol and voglibose), colesevelam, bromocriptine, insulin, metformin and thiazolidinediones (e.g. rosiglitazone, pioglitazone, troglitazone), among others.

In certain embodiments, the method of treatment includes one such therapy. In certain embodiments, the method of treatment includes a combination of two or more such therapies.

### The Biomarkers of the Invention

The one or more biomarkers (or sub-groups of such biomarkers) that are differentially present in a sample obtained from subjects with fibrosis as compared to normal subjects are listed in Tables 1 and 2. Thus, the one or more biomarkers of the present invention are useful in methods for diagnosing, prognosing, monitoring or determining the risk of fibrosis in a subject.

The one or more biomarkers (or sub-groups of such biomarkers) that are differentially present in a sample obtained from subjects with diabetes or a prediabetic condition as compared to normal subjects are listed in Tables 1 and 2. Thus, the one or more biomarkers of the present invention are useful in methods for diagnosing, prognosing, monitoring or determining the risk of diabetes or a prediabetic condition in a subject.

In particular, Table 1 provides the HUGO Gene Nomenclature Committee Gene Names (HGNC) and associated HGNC Gene ID's for the biomarkers of the present invention. Table 2 further provides a list of the UniProtKb accession numbers for the biomarkers of the present invention. As would be understood by a person skilled in the art, Table 1 provides information, such as the HGNC Gene ID, which can be used to determine the sequence of all of the RNA transcripts and thus all of the proteins which correspond to the biomarkers of the invention.

TABLE 1
HUGO Gene Nomenclature Committee Gene Names (HGNC) and
associated HGNC Gene ID's for the biomarkers of the present invention.

| | | | Gene | |
|---|---|---|---|---|
| No. | Name | HGNC_Symbol | ID | HGNC_official gene name |
| 1 | LYVE1 | LYVE1 | 10894 | lymphatic vessel endothelial hyaluronan |
| | | | | receptor 1 |
| 2 | AREG | AREG | 374 | amphiregulin |
| 3 | CD109 | CD109 | 135228 | CD109 molecule |
| 4 | ANXA3 | ANXA3 | 306 | annexin A3 |
| 5 | MYOF | MYOF | 26509 | myoferlin |
| 6 | IL18 | IL18 | 3606 | interleukin 18 (interferon-gamma-inducing |
| | | | | factor) |
| 7 | LIPH | LIPH | 200879 | lipase, member H |
| 8 | MRGPRX4 | MRGPRX4 | 117196 | MAS-related GPR, member X4 |
| 9 | VNN1 | VNN1 | 8876 | vanin 1 |
| 10 | ANXA1 | ANXA1 | 301 | annexin A1 |
| 11 | SERPINE2 | SERPINE2 | 5270 | serpin peptidase inhibitor, clade E (nexin, |
| | | | | plasminogen activator inhibitor type 1), |
| | | | | member 2 |
| 12 | SRPX2 | SRPX2 | 27286 | sushi-repeat containing protein, X-linked 2 |
| 13 | QPCT | QPCT | 25797 | glutaminyl-peptide cyclotransferase |
| 14 | F2RL1 | F2RL1 | 2150 | coagulation factor II (thrombin) receptor- |
| | | | | like 1 |
| 15 | KITLG | KITLG | 4254 | KIT ligand |
| 16 | MR1 | MR1 | 3140 | major histocompatibility complex, class I- |
| | | | | related |
| 17 | LY96 | LY96 | 23643 | lymphocyte antigen 96 |
| 18 | EMP3 | EMP3 | 2014 | epithelial membrane protein 3 |
| 19 | LAPTM5 | LAPTM5 | 7805 | lysosomal protein transmembrane 5 |
| 20 | SERPINB8 | SERPINB8 | 5271 | serpin peptidase inhibitor, clade B |
| | | | | (ovalbumin), member 8 |
| 21 | PLA2G2A | PLA2G2A | 5320 | phospholipase A2, group IIA (platelets, |
| | | | | synovial fluid) |
| 22 | CLDN6 | CLDN6 | 9074 | Claudin 6 |
| 23 | ANKRD1 | ANKRD1 | 27063 | ankyrin repeat domain 1 (cardiac muscle) |
| 24 | SLC16A4 | SLC16A4 | 9122 | solute carrier family 16, member 4 |
| | | | | (monocarboxylic acid transporter 5) |
| 25 | SUSD2 | SUSD2 | 56241 | sushi domain containing 2 |
| 26 | KLRC3 | KLRC3 | 3823 | iller cell lectin-like receptor subfamily C, |
| | | | | member 3 |
| 27 | C7 | C7 | 730 | complement component 7 |
| 28 | TRIM22 | TRIM22 | 10346 | tripartite motif containing 22 |
| 29 | GLIPR2 | GLIPR2 | 152007 | GLI pathogenesis-related 2 |
| 30 | SLFN5 | SLFN5 | 162394 | schlafen family member 5 |
| 31 | NRP1 | NRP1 | 8829 | neuropilin 1 |
| 32 | CRYAB | CRYAB | 1410 | crystallin, alpha B |
| 33 | MVP | MVP | 9961 | major vault protein |
| 34 | CD9 | CD9 | 928 | CD9 molecule |
| 35 | SPINT1 | SPINT1 | 6692 | serine peptidase inhibitor, Kunitz type 1 |
| 36 | OR52N4 | OR52N4 | 390072 | olfactory receptor, family 52, subfamily N, |
| | | | | member 4 |
| 37 | LTBP1 | LTBP1 | 4052 | latent transforming growth factor beta |
| | | | | binding protein 1 |
| 38 | ITGA3 | ITGA3 | 3675 | integrin, alpha 3 (antigen CD49C, alpha 3 |
| | | | | subunit of VLA-3 receptor) |
| 39 | KLRC2 | KLRC2 | 3822 | killer cell lectin-like receptor subfamily C, |
| | | | | member 2 |
| 40 | EMP1 | EMP1 | 2012 | epithelial membrane protein 1 |
| 41 | PLAU | PLAU | 5328 | plasminogen activator, urokinase |
| 42 | AXL | AXL | 558 | AXL receptor tyrosine kinase |
| 43 | LGALS1 | LGALS1 | 3956 | lectin, galactoside-binding, soluble, 1 |
| 44 | NAV3 | NAV3 | 89795 | neuron navigator 3 |
| 45 | CD3D | CD3D | 915 | CD3d molecule, delta (CD3-TCR |
| | | | | complex) |
| 46 | SAA4 | SAA4 | 6291 | serum amyloid A4, constitutive |
| 47 | SYT11 | SYT11 | 23208 | synaptotagmin XI |
| 48 | CHI3L1 | CHI3L1 | 1116 | chitinase 3-like 1 (cartilage glycoprotein- |
| | | | | 39) |
| 49 | SYTL2 | SYTL2 | 54843 | synaptotagmin-like 2 |
| 50 | GBA | GBA | 2629 | glucosidase, beta, acid |
| 51 | ABCC3 | ABCC3 | 8714 | ATP-binding cassette, sub-family C |
| | | | | (CFTR/MRP), member 3 |
| 52 | STX3 | STX3 | 6809 | syntaxin 3 |
| 53 | KLRC4 | KLRC4 | 8302 | killer cell lectin-like receptor subfamily C, |
| | | | | member 4 |
| 54 | PTAFR | PTAFR | 5724 | platelet-activating factor receptor |
| 55 | TAX1BP3 | TAX1BP3 | 30851 | Tax1 (human T-cell leukemia virus type I) |
| | | | | binding protein 3 |
| 56 | TMC7 | TMC7 | 79905 | transmembrane channel-like 7 |
| 57 | KLRK1 | KLRK1 | 22914 | killer cell lectin-like receptor subfamily K, |
| | | | | member 1 |
| 58 | KIAA1199 | KIAA1199 | 57214 | KIAA1199 |
| 59 | SPOCK2 | SPOCK2 | 9806 | sparc/osteonectin, cwcv and kazal-like |
| | | | | domains proteoglycan (testican) 2 |
| 60 | CD22 | CD22 | 933 | CD22 molecule |
| 61 | ITGA10 | ITGA10 | 8515 | integrin, alpha 10 |
| 62 | ARRDC4 | ARRDC4 | 91947 | arrestin domain containing 4 |
| 63 | C1S | C1S | 716 | complement component 1, s |
| | | | | subcomponent |
| 64 | PLEKHM1 | PLEKHM1 | 9842 | pleckstrin homology domain containing, |
| | | | | family M (with RUN domain) member 1 |
| 65 | ATP6V1D | ATP6V1D | 51382 | ATPase, H+ transporting, lysosomal |
| | | | | 34 kDa, V1 subunit D |
| 66 | TMED6 | TMED6 | 146456 | transmembrane emp24 protein transport |
| | | | | domain containing 6 |
| 67 | FAM135A | FAM135A | 57579 | family with sequence similarity 135, |
| | | | | member A |
| 68 | CD79A | CD79A | 973 | CD79a molecule, immunoglobulin- |
| | | | | associated alpha |
| 69 | IL31RA | IL31RA | 133396 | interleukin 31 receptor A |
| 70 | RHBDF2 | RHBDF2 | 79651 | rhomboid 5 homolog 2 (Drosophila) |
| 71 | HSPG2 | HSPG2 | 3339 | heparan sulfate proteoglycan 2 |
| 72 | LDLRAD1 | LDLRAD1 | 388633 | low density lipoprotein receptor class A |
| | | | | domain containing 1 |
| 73 | AAK1 | AAK1 | 22848 | AP2 associated kinase 1 |
| 74 | CD58 | CD58 | 965 | CD58 molecule |
| 75 | CSF1 | CSF1 | 1435 | colony stimulating factor 1 (macrophage) |
| 76 | KLF6 | KLF6 | 1316 | Kruppel-like factor 6 |
| 77 | LAMA3 | LAMA3 | 3909 | laminin, alpha 3 |
| 78 | TMEM104 | TMEM104 | 54868 | transmembrane protein 104 |
| 79 | SPARC | SPARC | 6678 | secreted protein, acidic, cysteine-rich |
| | | | | (osteonectin) |
| 80 | PVRL4 | PVRL4 | 81607 | poliovirus receptor-related 4 |
| 81 | LAD1 | LAD1 | 3898 | ladinin 1 |
| 82 | GLT25D2 | COLGALT2 | 23127 | collagen beta(1-O)galactosyltransferase 2 |
| 83 | TMEM9B | TMEM9B | 56674 | TMEM9 domain family, member B |
| 84 | CD59 | CD59 | 966 | CD59 molecule, complement regulatory |
| | | | | protein |
| 85 | CAPRIN2 | CAPRIN2 | 65981 | caprin family member 2 |
| 86 | FAM46A | FAM46A | 55603 | family with sequence similarity 46, |
| | | | | member A |
| 87 | ASB2 | ASB2 | 51676 | ankyrin repeat and SOCS box containing 2 |
| 88 | THBS1 | THBS1 | 7057 | thrombospondin 1 |
| 89 | CYR61 | CYR61 | 3491 | cysteine-rich, angiogenic inducer, 61 |
| 90 | THBS3 | THBS3 | 7059 | thrombospondin 3 |
| 91 | PDGF | PDGFRA | 5156 | platelet-derived growth factor receptor, |
| | | | | alpha polypeptide |
| 92 | TGFB1 | TGFB1 | 7040 | transforming growth factor, beta 1 |
| 93 | PHLDA3 | PHLDA3 | 23612 | pleckstrin homology-like domain, family A, |
| | | | | member 3 |
| 94 | DDIT4 | DDIT4 | 54541 | DNA-damage-inducible transcript 4 |
| 95 | G6PC | G6PC | 2538 | Glucose-6-phosphatase, catalytic subunit |
| 96 | PGM2L1 | PGM2L1 | 283209 | Phosphoglucomutase 2-like 1 |
| 97 | C14orf105 | C14orf105 | 55195 | Chromosome 14 open reading frame 105 |
| 98 | CTSE | CTSE | 1510 | Cathepsin E |
| 99 | FAS | FAS | 355 | Fas cell surface death receptor |
| 100 | PRF1 | PRF1 | 5551 | Perforin 1 (pore forming protein) |
| 101 | CAPN | CAPN | 824 | Calpain 2, (m/II) large subunit |
| 102 | HAL | HAL | 3034 | Histidine ammonia-lysase |

TABLE 2
UniProtKb accession numbers for the biomarkers of the present
invention.

| | | | UniProtKB | |
|---|---|---|---|---|
| No. | Name | HGNC_Symbol | Accession | Protein Name |
| 1 | LYVE1 | LYVE1 | Q9Y5Y7 | Lymphatic vessel endothelial |
| | | | | hyaluronic acid receptor 1 |
| 2 | AREG | AREG | P15514 | amphiregulin |
| 3 | CD109 | CD109 | Q6YHK3 | CD109 antigen |
| 4 | ANXA3 | ANXA3 | P12429 | Annexin A3 |
| 5 | MYOF | MYOF | Q9NZM1 | Myoferlin |
| 6 | IL18 | IL18 | Q14116 | Interleukin-18 |
| 7 | LIPH | LIPH | Q8WWY8 | Lipase member H |
| 8 | MRGPRX4 | MRGPRX4 | Q96LA9 | Mas-related G-protein coupled |
| | | | | receptor member X4 |
| 9 | VNN1 | VNN1 | O95497 | Pantetheinase |
| 10 | ANXA1 | ANXA1 | P04083 | Annexin A1 |
| 11 | SERPINE2 | SERPINE2 | P07093 | Glia-derived nexin |
| 12 | SRPX2 | SRPX2 | O60687 | Sushi repeat-containing protein |
| 12 | SRPX2 | SRPX2 | O60687 | SRPX2 |
| 13 | QPCT | QPCT | Q16769 | Glutaminyl-peptide |
| | | | | cyclotransferase |
| 14 | F2RL1 | F2RL1 | P55085 | Proteinase-activated receptor 2 |
| 15 | KITLG | KITLG | P21583 | Kit ligand |
| 16 | MR1 | MR1 | Q95460 | Major histocompatibility complex |
| | | | | class I-related gene protein |
| 17 | LY96 | LY96 | Q9Y6Y9 | Lymphocyte antigen 96 |
| 18 | EMP3 | EMP3 | P54852 | Epithelial membrane protein 3 |
| 19 | LAPTM5 | LAPTM5 | Q13571 | Lysosomal-associated |
| | | | | transmembrane protein 5 |
| 20 | SERPINB8 | SERPINB8 | P50452 | Serpin B8 |
| 21 | PLA2G2A | PLA2G2A | P14555 | Phospholipase A2, membrane |
| | | | | associated |
| 22 | CLDN6 | CLDN6 | P56747 | Claudin-6 |
| 23 | ANKRD1 | ANKRD1 | Q15327 | Ankyrin repeat domain-containing |
| | | | | protein 1 |
| 24 | SLC16A4 | SLC16A4 | O15374 | Monocarboxylate transporter 5 |
| 25 | SUSD2 | SUSD2 | Q9UGT4 | Sushi domain-containing protein 2 |
| 26 | KLRC3 | KLRC3 | Q07444 | NKG2-E type II integral |
| | | | | membrane protein |
| 27 | C7 | C7 | P10643 | Complement component C7 |
| 28 | TRIM22 | TRIM22 | Q8IYM9 | E3 ubiquitin-protein ligase |
| | | | | TRIM22 |
| 29 | GLIPR2 | GLIPR2 | Q9H4G4 | Golgi-associated plant |
| | | | | pathogenesis-related protein 1 |
| 30 | SLFN5 | SLFN5 | Q08AF3 | Schlafen family member 5 |
| 31 | NRP1 | NRP1 | O14786 | Neuropilin-1 |
| 32 | CRYAB | CRYAB | P02511 | Alpha-crystallin B chain |
| 33 | MVP | MVP | Q14764 | Major vault protein |
| 34 | CD9 | CD9 | P21926 | CD9 antigen |
| 35 | SPINT1 | SPINT1 | O43278 | Kunitz-type protease inhibitor 1 |
| 36 | OR52N4 | OR52N4 | Q8NGI2 | Olfactory receptor 52N4 |
| 37 | LTBP1 | LTBP1 | Q14766 | Latent-transforming growth factor |
| | | | | beta-binding protein 1 |
| 38 | ITGA3 | ITGA3 | P26006 | Integrin alpha-3 |
| 39 | KLRC2 | KLRC2 | P26717 | NKG2-C type II integral |
| | | | | membrane protein |
| 40 | EMP1 | EMP1 | P54849 | Epithelial membrane protein 1 |
| 41 | PLAU | PLAU | P00749 | Urokinase-type plasminogen |
| | | | | activator |
| 42 | AXL | AXL | P30530 | Tyrosine-protein kinase receptor |
| | | | | UFO |
| 43 | LGALS1 | LGALS1 | P09382 | Galectin-1 |
| 44 | NAV3 | NAV3 | Q8IVL0 | Neuron navigator 3 |
| 45 | CD3D | CD3D | P04234 | T-cell surface glycoprotein CD3 |
| | | | | delta chain |
| 46 | SAA4 | SAA4 | P35542 | Serum amyloid A-4 protein |
| 47 | SYT11 | SYT11 | Q9BT88 | Synaptotagmin-11 |
| 48 | CHI3L1 | CHI3L1 | P36222 | Chitinase-3-like protein 1 |
| 49 | SYTL2 | SYTL2 | Q9HCH5 | Synaptotagmin-like protein 2 |
| 50 | GBA | GBA | P04062 | Glucosylceramidase |
| 51 | ABCC3 | ABCC3 | O15438 | Canalicular multispecific organic |
| | | | | anion transporter 2 |
| 52 | STX3 | STX3 | Q13277 | Syntaxin-3 |
| 53 | KLRC4 | KLRC4 | O43908 | NKG2-F type II integral |
| | | | | membrane protein |
| 54 | PTAFR | PTAFR | P25105 | Platelet-activating factor receptor |
| 55 | TAX1BP3 | TAX1BP3 | O14907 | Tax1-binding protein 3 |
| 56 | TMC7 | TMC7 | Q7Z402 | Transmembrane channel-like |
| | | | | protein 7 |
| 57 | KLRK1 | KLRK1 | P26718 | NKG2-D type II integral |
| | | | | membrane protein |
| 58 | KIAA1199 | KIAA1199 | Q8WUJ3 | Protein KIAA1199 |
| 59 | SPOCK2 | SPOCK2 | Q92563 | Testican-2 |
| 60 | CD22 | CD22 | P20273 | B-cell receptor CD22 |
| 61 | ITGA10 | ITGA10 | O75578 | Integrin alpha-10 |
| 62 | ARRDC4 | ARRDC4 | Q8NCT1 | Arrestin domain-containing |
| | | | | protein 4 |
| 63 | C1S | C1S | P09871 | Complement C1s subcomponent |
| 64 | PLEKHM1 | PLEKHM1 | Q9Y4G2 | Pleckstrin homology domain- |
| | | | | containing family M member 1 |
| 65 | ATP6V1D | ATP6V1D | Q9Y5K8 | V-type proton ATPase subunit D |
| 66 | TMED6 | TMED6 | Q8WW62 | Transmembrane emp24 domain- |
| | | | | containing protein 6 |
| 67 | FAM135A | FAM135A | Q9P2D6 | Protein FAM135A |
| 68 | CD79A | CD79A | P11912 | B-cell antigen receptor complex- |
| | | | | associated protein alpha chain |
| 69 | IL31RA | IL31RA | Q8NI17 | Interleukin-31 receptor subunit |
| | | | | alpha |
| 70 | RHBDF2 | RHBDF2 | Q6PJF5 | Inactive rhomboid protein 2 |
| 71 | HSPG2 | HSPG2 | P98160 | Basement membrane-specific |
| | | | | heparan sulfate proteoglycan |
| | | | | core protein |
| 72 | LDLRAD1 | LDLRAD1 | Q5T700 | Low-density lipoprotein receptor |
| | | | | class A domain-containing |
| | | | | protein 1 |
| 73 | AAK1 | AAK1 | Q2M2I8 | AP2-associated protein kinase 1 |
| 74 | CD58 | CD58 | P19256 | Lymphocyte function-associated |
| | | | | antigen 3 |
| 75 | CSF1 | CSF1 | P09603 | Macrophage colony-stimulating |
| | | | | factor 1 |
| 76 | KLF6 | KLF6 | Q99612 | Krueppel-like factor 6 |
| 77 | LAMA3 | LAMA3 | Q16787 | Laminin subunit alpha-3 |
| 78 | TMEM104 | TMEM104 | Q8NE00 | Transmembrane protein 104 |
| 79 | SPARC | SPARC | P09486 | SPARC |
| 80 | PVRL4 | PVRL4 | Q96NY8 | Poliovirus receptor-related |
| | | | | protein 4 |
| 81 | LAD1 | LAD1 | O00515 | Ladinin-1 |
| 82 | GLT25D2 | COLGALT2 | Q8IYK4 | Procollagen |
| | | | | galactosyltransferase 2 |
| 83 | TMEM9B | TMEM9B | Q9NQ34 | Transmembrane protein 9B |
| 84 | CD59 | CD59 | P13987 | CD59 glycoprotein |
| 85 | CAPRIN2 | CAPRIN2 | Q6IMN6 | Caprin-2 |
| 86 | FAM46A | FAM46A | Q96IP4 | Protein FAM46A |
| 87 | ASB2 | ASB2 | Q96Q27 | Ankyrin repeat and SOCS box |
| | | | | protein 2 |
| 88 | THBS1 | THBS1 | P07996 | Thrombospondin-1 |
| 89 | CYR61 | CYR61 | O00622 | Protein CYR61 |
| 90 | THBS3 | THBS3 | P49746 | Thrombospondin-3 |
| 91 | PDGF | PDGFRA | P16234 | Platelet-derived growth factor |
| | | | | receptor alpha |
| 92 | TGFB1 | TGFB1 | P01137 | Transforming growth factor beta-1 |
| 93 | PHLDA3 | PHLDA3 | Q9Y5J5 | Pleckstrin homology-like domain |
| | | | | family A member 3 |
| 94 | DDIT4 | DDIT4 | Q9NX09 | DNA damage-inducible transcript |
| | | | | 4 protein |
| 95 | G6PC | G6PC | P35575 | Glucose-6-phosphatase |
| 96 | PGM2L1 | PGM2L1 | Q6PCE3 | Glucose 1,6-biphosphate |
| | | | | synthase |
| 97 | C14orf105 | C14orf105 | Q9NVL8 | Uncharacterised protein |
| | | | | C14orf105 |
| 98 | CTSE | CTSE | P14091 | Cathepsin E |
| 99 | FAS | FAS | P25445 | Tumor necrosis factor receptor |
| | | | | superfamily member 6 |
| 100 | PRF1 | PRF1 | P14222 | Perforin-1 |
| 101 | CAPN | CAPN | P17655 | Calpain-2 catalytic subunit |
| 102 | HAL | HAL | P42357 | Histidine ammonia-lysase |

In one embodiment, the one or more biomarkers are selected from the group consisting of CSF1, LYVE1, MRGPRX4, IL-18, CYR61, G6PC, MVP, PGM2L1, ANXA3, SERPINB8, MYOF and SPARC (as defined in Tables 1 and 2). Any combination of 1, 2, 3, 4, 5, 6, 7, 8, 9, 10, 11 or 12 of these biomarkers is encompassed by the present invention.

In another embodiment, the one or more biomarkers are selected from the group consisting of CSF1, LYVE1, MRGPRX4, IL-18 and CYR61 (as defined in Tables 1 and 2). Any combination of 1, 2, 3, 4 or 5 of these biomarkers is encompassed by the present invention. In one embodiment, an increase in the effective amount of any of these biomarkers indicates that the patient has, or may be at risk of having, fibrosis, e.g. liver fibrosis. In another embodiment, an increase in the effective amount of any of these biomarkers indicates that the patient has, or may be at risk of having, diabetes or a prediabetic condition.

In another embodiment, the one or more biomarkers are selected from the group consisting of G6PC, MVP, PGM2L1, ANXA3, SERPINB8, MYOF and SPARC. Any combination of 1, 2, 3, 4, 5, 6 or 7 of these biomarkers is encompassed by the present invention. In one embodiment, a decrease in the effective amount of any of these biomarkers indicates that the patient has, or may be at risk of having, fibrosis, e.g. liver fibrosis. In another embodiment, a decrease in the effective amount of any of these biomarkers indicates that the patient has, or may be at risk of having, diabetes or a prediabetic condition.

In one embodiment, the biomarker is a nucleic acid (e.g., DNA, such as cDNA or amplified DNA, or RNA, such as mRNA). In another embodiment, the biomarker is a protein. As used herein, the terms “protein”, “peptide”, and “polypeptide” are, unless otherwise indicated, interchangeable. When the effective amounts of two or more biomarkers are determined, the biomarkers may all be protein biomarkers or all nucleic acid biomarkers. Alternatively, the biomarkers may be both protein and nucleic acid biomarkers.

The present invention also encompasses, without limitation, polymorphisms, isoforms, metabolites, mutants, variants, derivatives, modifications, subunits, fragments, protein-ligand complexes and degradation products of the biomarkers listed in Tables 1 and 2.

The protein fragments can be 200, 150, 100, 50, 25, 10 amino acids or fewer in length. The nucleic acid fragments can be 1000, 500, 250 150, 100, 50, 25, 10 nucleotides or fewer in length.

Variants of the protein biomarkers of the present invention include polypeptides with altered amino acid sequences due to amino acid substitutions, deletions, or insertions. Variant polypeptides may comprise conservative or non-conservative amino acid substitutions, deletions or additions. Variants include polypeptides that have an amino acid sequence being at least 70%, at least 80%, at least 90%, at least 95%, at least 98% or at least 99% identical to the amino acid sequences of the polypeptides listed in Tables 1 and 2. Variants may be allelic variants, splice variants or any other species specific homologs, paralogs, or orthologs.

Derivatives of the protein biomarkers of the present invention are polypeptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

In one embodiment, the present invention provides methods for diagnosing, prognosing or monitoring fibrosis, e.g. liver fibrosis, based on the determination of the effective amount of one or more nucleic acid biomarkers (e.g. a nucleic acid expressed from a gene listed in Table 1 or Table 2). Such nucleic acid biomarkers can be, for example, mRNA transcripts, cDNA or some other nucleic acid. Accordingly, determining the effective amount (and changes in the effective amount) of such nucleic acids can be used in the diagnosis, prognosis and monitoring of fibrosis.

In one embodiment, the present invention provides methods for diagnosing, prognosing or monitoring diabetes or a prediabetic condition based on the determination of the effective amount of one or more nucleic acid biomarkers (e.g. a nucleic acid expressed from a gene listed in Table 1 or Table 2). Such nucleic acid biomarkers can be, for example, mRNA transcripts, cDNA or some other nucleic acid. Accordingly, determining the effective amount (and changes in the effective amount) of such nucleic acids can be used in the diagnosis, prognosis and monitoring of diabetes or a prediabetic condition.

In one embodiment, the present invention provides methods for diagnosing, prognosing or monitoring fibrosis, e.g. liver fibrosis, based on the determination of the effective amount of one or more protein biomarkers (e.g. a protein expressed from a gene listed in Table 1 or Table 2). Changes associated with many disease states are reflected in the effective amount of the one or more protein biomarkers in the blood, as well as other bodily fluids, such as urine. Accordingly, determining the effective amount (and changes in the effective amount) of such proteins can be used in the diagnosis, prognosis and monitoring of fibrosis.

In one embodiment, the present invention provides methods for diagnosing, prognosing or monitoring diabetes or a prediabetic condition based on the determination of the effective amount of one or more protein biomarkers (e.g. a protein expressed from a gene listed in Table 1 or Table 2). Changes associated with many disease states are reflected in the effective amount of the one or more protein biomarkers in the blood, as well as other bodily fluids, such as urine. Accordingly, determining the effective amount (and changes in the effective amount) of such proteins can be used in the diagnosis, prognosis and monitoring of diabetes or a prediabetic condition.

In one embodiment, the present invention provides methods for diagnosing, prognosing or monitoring fibrosis, e.g. liver fibrosis, based on the determination of the effective amount of one or more, wherein said one or more biomarkers are selected from the group consisting of MVP, PGM2L1, ANXA3, MYOF, LYVE1, SERBINB8, IL18, MRGPRX4, G6PC, DDIT4, CSF1 and SPARC (as defined in Tables 1 and 2).

In one embodiment, the present invention provides methods for diagnosing, prognosing or monitoring diabetes or a prediabetic condition based on the determination of the effective amount of one or more biomarkers, wherein said one or more biomarkers are selected from the group consisting of CYR61, MVP, PGM2L1, ANXA3, MYOF, LYVE1, SERBINB8, IL18, MRGPRX4, G6PC, DDIT4, and CSF1 (as defined in Tables 1 and 2).

In one embodiment, the present invention provides methods for diagnosing, prognosing or monitoring fibrosis based on the determination of the effective amount of one or more biomarkers, wherein said one or more biomarkers are selected from the group consisting of CSF1, LYVE1, MRGPRX4 and IL-18 (as defined in Tables 1 and 2).

In one embodiment, the present invention provides methods for diagnosing, prognosing or monitoring diabetes or a prediabetic condition based on the determination of the effective amount of one or more biomarkers, wherein said one or more biomarkers are selected from the group consisting of CSF1, MRGPRX4, IL-18, CYR61 and LYVE1 (as defined in Tables 1 and 2).

In one embodiment, the present invention provides methods for diagnosing, prognosing or monitoring fibrosis based on the determination of the effective amount of one or more biomarkers, wherein said one or more biomarkers are selected from the group consisting of G6PC, MVP, PGM2L1, ANXA3, SERPINB8, MYOF and SPARC (as defined in Tables 1 and 2).

In one embodiment, the present invention provides methods for diagnosing, prognosing or monitoring diabetes or a prediabetic condition based on the determination of the effective amount of one or more biomarkers, wherein said one or more biomarkers are selected from the group consisting of G6PC, MVP, PGM2L1, ANXA3, SERPINB8 and MYOF (as defined in Tables 1 and 2).

In one embodiment, the present invention provides methods for diagnosing, prognosing or monitoring fibrosis based on the determination of the effective amount of one or more biomarkers, wherein said one or more biomarkers are selected from the group consisting of C14orf105, CD109, CTSE, F2RL1, FAS, LTBP1, KLRC3, MR1, SUSD2, TRIM22, PRF1, C7, CAPN2, NRP1, QPCT, SRPX2 and HAL (as defined in Tables 1 and 2).

In one embodiment, the present invention provides methods for diagnosing, prognosing or monitoring diabetes or a prediabetic condition based on the determination of the effective amount of one or more biomarkers, wherein said one or more biomarkers are selected from the group consisting of C14orf105, CD109, CTSE, F2RL1, FAS, LTBP1, KLRC3, MR1, SUSD2, TRIM22, PRF1, C7, CAPN2, NRP1, QPCT, SRPX2 and HAL (as defined in Tables 1 and 2).

In one embodiment, the present invention provides methods for diagnosing, prognosing or monitoring fibrosis based on the determination of the effective amount of one or more biomarkers, wherein said one or more biomarkers are selected from the group consisting of SPARC, MVP, PGM2L1, ANXA3, MYOF, LYVE1, SERBINB8, IL18, MRGPRX4, G6PC, DDIT4, CSF1, C14orf105, CD109, CTSE, F2RL1, FAS, LTBP1, KLRC3, MR1, SUSD2, TRIM22, PRF1, C7, CAPN2, NRP1, QPCT, SRPX2 and HAL (as defined in Tables 1 and 2).

In one embodiment, the present invention provides methods for diagnosing, prognosing or monitoring diabetes or a prediabetic condition based on the determination of the effective amount of one or more biomarkers, wherein said one or more biomarkers are selected from the group consisting of CYR61, MVP, PGM2L1, ANXA3, MYOF, LYVE1, SERBINB8, IL18, MRGPRX4, G6PC, DDIT4, CSF1, C14orf105, CD109, CTSE, F2RL1, FAS, LTBP1, KLRC3, MR1, SUSD2, TRIM22, PRF1, C7, CAPN2, NRP1, QPCT, SRPX2 and HAL (as defined in Tables 1 and 2).

In one embodiment, the present invention provides methods for diagnosing, prognosing or monitoring diabetes or a prediabetic condition based on the determination of the effective amount of one or more biomarkers that are associated with immune response and/or inflammation, wherein said one or more biomarkers are selected from the group consisting of AREG, IL18, VNN1, ANXA1, LY96, PLA2G2A, C7, SAA4, PTAFR, KLRK1, C1S, IL31RA, CSF1 and KLF6 (as defined in Tables 1 and 2).

In one embodiment, the present invention provides methods for diagnosing, prognosing or monitoring fibrosis based on the determination of the effective amount of one or more biomarkers that are associated with immune response and/or inflammation, wherein said one or more biomarkers are selected from the group consisting of AREG, IL18, VNN1, ANXA1, LY96, PLA2G2A, C7, SAA4, PTAFR, KLRK1, C1 S, IL31RA, CSF1 and KLF6 (as defined in Tables 1 and 2).

In one embodiment, the present invention provides methods for diagnosing, prognosing or monitoring diabetes or a prediabetic condition based on the determination of the effective amount of one or more biomarkers that are associated with extracellular matrix maintenance or composition, wherein said one or more biomarkers are selected from the group consisting of LYVE, LTBP1, ITGA3, LGALS1, CHI3L1, SPOCK2, ITGA10, HSPG2, LAMA3, SPARC, LAD1, CYR61, THBS3, PDGF, and TGFB1 (as defined in Tables 1 and 2).

In one embodiment, the present invention provides methods for diagnosing, prognosing or monitoring fibrosis based on the determination of the effective amount of one or more biomarkers that are associated with extracellular matrix maintenance or composition, wherein said one or more biomarkers are selected from the group consisting of LYVE, LTBP1, ITGA3, LGALS1, CHI3L1, SPOCK2, ITGA10, HSPG2, LAMA3, SPARC, LAD1, CYR61, THBS3, PDGF, and TGFB1 (as defined in Tables 1 and 2).

In one embodiment, the present invention provides methods for diagnosing, prognosing or monitoring diabetes or a prediabetic condition based on the determination of the effective amount of one or more biomarkers that are associated with metabolism, wherein said one or more biomarkers are selected from the group consisting of LYVE, AREG, IL18, VNN1, KITLG, PLA2G2A, ANKRD1, TRIM22, AXL, CHI3L1, GBA, PTAFR, KLRK1, ATPGV1D, IL31RA, AAK1, CSF1 and GLT25D2 (as defined in Tables 1 and 2).

In one embodiment, the present invention provides methods for diagnosing, prognosing or monitoring fibrosis based on the determination of the effective amount of one or more biomarkers that are associated with metabolism, wherein said one or more biomarkers are selected from the group consisting of LYVE, AREG, IL18, VNN1, KITLG, PLA2G2A, ANKRD1, TRIM22, AXL, CHI3L1, GBA, PTAFR, KLRK1, ATPGV1D, IL31RA, AAK1, CSF1 and GLT25D2 (as defined in Tables 1 and 2).

In one embodiment, the present invention provides methods for diagnosing, prognosing or monitoring diabetes or a prediabetic condition based on the determination of the effective amount of one or more biomarkers that are associated with response to stress, wherein said one or more biomarkers are selected from the group consisting of MYOF, VNN1 and CRYAB (as defined in Tables 1 and 2).

In one embodiment, the present invention provides methods for diagnosing, prognosing or monitoring fibrosis based on the determination of the effective amount of one or more biomarkers that are associated with response to stress, wherein said one or more biomarkers are selected from the group consisting of MYOF, VNN1 and CRYAB (as defined in Tables 1 and 2).

In one embodiment, the present invention provides methods for diagnosing, prognosing or monitoring diabetes or a prediabetic condition based on the determination of the effective amount of one or more biomarkers selected from the group consisting of SPARC, CYR61, PDGF and TGFB1 (as defined in Tables 1 and 2).

In one embodiment, the present invention provides methods for diagnosing, prognosing or monitoring fibrosis based on the determination of the effective amount of one or more biomarkers selected from the group consisting of SPARC, CYR61, PDGF and TGFB1 (as defined in Tables 1 and 2).

Thus, in one embodiment, the methods of the present invention comprise determining the effective amount of LYVE1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, LYVE1 and the other biomarkers are proteins. In one embodiment, LYVE1 and the other biomarkers are nucleic acids.

In one embodiment, the methods of the present invention comprise determining the effective amount of AREG (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, AREG and the other biomarkers are proteins. In one embodiment, AREG and the other biomarkers are nucleic acids.

In one embodiment, the methods of the present invention comprise determining the effective amount of CD109 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CD109 and the other biomarkers are proteins. In one embodiment, CD109 and the other biomarkers are nucleic acids.

In one embodiment, the methods of the present invention comprise determining the effective amount of ANXA3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, ANXA3 and the other biomarkers are proteins. In one embodiment, ANXA3 and the other biomarkers are nucleic acids.

In one embodiment, the methods of the present invention comprise determining the effective amount of MYOF (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, MYOF and the other biomarkers are proteins. In one embodiment, MYOF and the other biomarkers are nucleic acids expressed from a gene listed in Table 1 or Table 2.

In one embodiment, the methods of the present invention comprise determining the effective amount of ID 8 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, IL18 and the other biomarkers are proteins. In one embodiment, IL18 and the other biomarkers are nucleic acids.

In one embodiment, the methods of the present invention comprise determining the effective amount of LIPH (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, LIPH and the other biomarkers are proteins. In one embodiment, LIPH and the other biomarkers are nucleic acids.

In one embodiment, the methods of the present invention comprise determining the effective amount of MRGPRX4 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, MRGPRX4 and the other biomarkers are proteins. In one embodiment, MRGPRX4 and the other biomarkers are nucleic acids.

In one embodiment, the methods of the present invention comprise determining the effective amount of VNN1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, VNN1 and the other biomarkers are proteins. In one embodiment, VNN1 and the other biomarkers are nucleic acids.

In one embodiment, the methods of the present invention comprise determining the effective amount of ANXA1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, ANXA1 and the other biomarkers are proteins. In one embodiment, ANXA1 and the other biomarkers are nucleic acids.

In one embodiment, the methods of the present invention comprise determining the effective amount of SERPINE2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SERPINE2 and the other biomarkers are proteins. In one embodiment, SERPINE2 and the other biomarkers are nucleic acids.

In one embodiment, the methods of the present invention comprise determining the effective amount of SRPX2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SRPX2 and the other biomarkers are proteins. In one embodiment, SRPX2 and the other biomarkers are nucleic acids.

In one embodiment, the methods of the present invention comprise determining the effective amount of QPCT (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, QPCT and the other biomarkers are proteins. In one embodiment, QPCT and the other biomarkers are nucleic acids.

In one embodiment, the methods of the present invention comprise determining the effective amount of F2RL1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, F2RL1 and the other biomarkers are proteins. In one embodiment, F2RL1 and the other biomarkers are nucleic acids.

In one embodiment, the methods of the present invention comprise determining the effective amount of KITLG (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, KITLG and the other biomarkers are proteins. In one embodiment, KITLG and the other biomarkers are nucleic acids.

In one embodiment, the methods of the present invention comprise determining the effective amount of MR1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, MR1 and the other biomarkers are proteins. In one embodiment, MR1 and the other biomarkers are nucleic acids.

In one embodiment, the methods of the present invention comprise determining the effective amount of LY96 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, LY96 and the other biomarkers are proteins. In one embodiment, LY96 and the other biomarkers are nucleic acids.

In one embodiment, the methods of the present invention comprise determining the effective amount of EMP3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, EMP3 and the other biomarkers are proteins. In one embodiment, EMP3 and the other biomarkers are nucleic acids.

In one embodiment, the methods of the present invention comprise determining the effective amount of LAPTM5 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, LAPTM5 and the other biomarkers are proteins. In one embodiment, LAPTM5 and the other biomarkers are nucleic acids.

In one embodiment, the methods of the present invention comprise determining the effective amount of SERPINB8 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SERPINB8 and the other biomarkers are proteins. In one embodiment, SERPINB8 and the other biomarkers are nucleic acids.

In one embodiment, the methods of the present invention comprise determining the effective amount of PLA2G2A (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, PLA2G2A and the other biomarkers are proteins. In one embodiment, PLA2G2A and the other biomarkers are nucleic acids.

In one embodiment, the methods of the present invention comprise determining the effective amount of CLDN6 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CLDN6 and the other biomarkers are proteins. In one embodiment, CLDN6 and the other biomarkers are nucleic acids.

In one embodiment, the methods of the present invention comprise determining the effective amount of ANKRD1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, ANKRD1 and the other biomarkers are proteins. In one embodiment, ANKRD1 and the other biomarkers are nucleic acids.

In one embodiment, the methods of the present invention comprise determining the effective amount of SLC16A4 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SLC16A4 and the other biomarkers are proteins. In one embodiment, SLC16A4 and the other biomarkers are nucleic acids.

In one embodiment, the methods of the present invention comprise determining the effective amount of SUSD2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SUSD2 and the other biomarkers are proteins. In one embodiment, SUSD2 and the other biomarkers are nucleic acids.

In one embodiment, the methods of the present invention comprise determining the effective amount of KLRC3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, KLRC3 and the other biomarkers are proteins. In one embodiment, KLRC3 and the other biomarkers are nucleic acids.

In one embodiment, the methods of the present invention comprise determining the effective amount of C7 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, C7 and the other biomarkers are proteins. In one embodiment, C7 and the other biomarkers are nucleic acids.

In one embodiment, the methods of the present invention comprise determining the effective amount of TRIM22 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, TRIM22 and the other biomarkers are proteins. In one embodiment, TRIM22 and the other biomarkers are nucleic acids.

In one embodiment, the methods of the present invention comprise determining the effective amount of GLIPR2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, GLIPR2 and the other biomarkers are proteins. In one embodiment, GLIPR2 and the other biomarkers are nucleic acids.

In one embodiment, the methods of the present invention comprise determining the effective amount of SLFN5 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SLFN5 and the other biomarkers are proteins. In one embodiment, SLFN5 and the other biomarkers are nucleic acids.

In one embodiment, the methods of the present invention comprise determining the effective amount of NRP1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, NRP1 and the other biomarkers are proteins. In one embodiment, NRP1 and the other biomarkers are nucleic acids.

In one embodiment, the methods of the present invention comprise determining the effective amount of CRYAB (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CRYAB and the other biomarkers are proteins. In one embodiment, CRYAB and the other biomarkers are nucleic acids.

In one embodiment, the methods of the present invention comprise determining the effective amount of MVP (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, MVP and the other biomarkers are proteins. In one embodiment, MVP and the other biomarkers are nucleic acids.

In one embodiment, the methods of the present invention comprise determining the effective amount of CD9 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CD9 and the other biomarkers are proteins. In one embodiment, CD9 and the other biomarkers are nucleic acids.

In one embodiment, the methods of the present invention comprise determining the effective amount of SPINT1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SPINT1 and the other biomarkers are proteins. In one embodiment, SPINT1 and the other biomarkers are nucleic acids.

In one embodiment, the methods of the present invention comprise determining the effective amount of OR52N4 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, OR52N4 and the other biomarkers are proteins. In one embodiment, OR52N4 and the other biomarkers are nucleic acids.

In one embodiment, the methods of the present invention comprise determining the effective amount of LTBP1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, LTBP1 and the other biomarkers are proteins. In one embodiment, LTBP1 and the other biomarkers are nucleic acids.

In one embodiment, the methods of the present invention comprise determining the effective amount of ITGA3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, ITGA3 and the other biomarkers are proteins. In one embodiment, ITGA3 and the other biomarkers are nucleic acids.

In one embodiment, the methods of the present invention comprise determining the effective amount of KLRC2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, KLRC2 and the other biomarkers are proteins. In one embodiment, KLRC2 and the other biomarkers are nucleic acids.

In one embodiment, the methods of the present invention comprise determining the effective amount of EMP1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, EMP1 and the other biomarkers are proteins. In one embodiment, EMP1 and the other biomarkers are nucleic acids.

In one embodiment, the methods of the present invention comprise determining the effective amount of PLAU (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, PLAU and the other biomarkers are proteins. In one embodiment, PLAU and the other biomarkers are nucleic acids.

In one embodiment, the methods of the present invention comprise determining the effective amount of AXL (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, AXL and the other biomarkers are proteins. In one embodiment, AXL and the other biomarkers are nucleic acids.

In one embodiment, the methods of the present invention comprise determining the effective amount of LGALS1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, LGALS1 and the other biomarkers are proteins. In one embodiment, LGALS1 and the other biomarkers are nucleic acids.

In one embodiment, the methods of the present invention comprise determining the effective amount of NAV3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, NAV3 and the other biomarkers are proteins. In one embodiment, NAV3 and the other biomarkers are nucleic acids.

In one embodiment, the methods of the present invention comprise determining the effective amount of CD3D (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CD3D and the other biomarkers are proteins. In one embodiment, CD3D and the other biomarkers are nucleic acids.

In one embodiment, the methods of the present invention comprise determining the effective amount of SAA4 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SAA4 and the other biomarkers are proteins. In one embodiment, SAA4 and the other biomarkers are nucleic acids.

In one embodiment, the methods of the present invention comprise determining the effective amount of SYT11 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SYT11 and the other biomarkers are proteins. In one embodiment, SYT11 and the other biomarkers are nucleic acids.

In one embodiment, the methods of the present invention comprise determining the effective amount of CHI3L1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CHI3L1 and the other biomarkers are proteins. In one embodiment, CHI3L1 and the other biomarkers are nucleic acids.

In one embodiment, the methods of the present invention comprise determining the effective amount of SYTL2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SYTL2 and the other biomarkers are proteins. In one embodiment, SYTL2 and the other biomarkers are nucleic acids.

In one embodiment, the methods of the present invention comprise determining the effective amount of GBA (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, GBA and the other biomarkers are proteins. In one embodiment, GBA and the other biomarkers are nucleic acids.

In one embodiment, the methods of the present invention comprise determining the effective amount of ABCC3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, ABCC3 and the other biomarkers are proteins. In one embodiment, ABCC3 and the other biomarkers are nucleic acids.

In one embodiment, the methods of the present invention comprise determining the effective amount of STX3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, STX3 and the other biomarkers are proteins. In one embodiment, STX3 and the other biomarkers are nucleic acids.

In one embodiment, the methods of the present invention comprise determining the effective amount of KLRC4 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, KLRC4 and the other biomarkers are proteins. In one embodiment, KLRC4 and the other biomarkers are nucleic acids.

In one embodiment, the methods of the present invention comprise determining the effective amount of PTAFR (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, PTAFR and the other biomarkers are proteins. In one embodiment, PTAFR and the other biomarkers are nucleic acids.

In one embodiment, the methods of the present invention comprise determining the effective amount of TAX1BP3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, TAX1BP3 and the other biomarkers are proteins. In one embodiment, TAX1BP3 and the other biomarkers are nucleic acids.

In one embodiment, the methods of the present invention comprise determining the effective amount of TMC7 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, TMC7 and the other biomarkers are proteins. In one embodiment, TMC7 and the other biomarkers are nucleic acids.

In one embodiment, the methods of the present invention comprise determining the effective amount of KLRK1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, KLRK1 and the other biomarkers are proteins. In one embodiment, KLRK1 and the other biomarkers are nucleic acids.

In one embodiment, the methods of the present invention comprise determining the effective amount of KIAA1199 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, KIAA1199 and the other biomarkers are proteins. In one embodiment, KIAA1199 and the other biomarkers are nucleic acids.

In one embodiment, the methods of the present invention comprise determining the effective amount of SPOCK2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SPOCK2 and the other biomarkers are proteins. In one embodiment, SPOCK2 and the other biomarkers are nucleic acids.

In one embodiment, the methods of the present invention comprise determining the effective amount of CD22 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CD22 and the other biomarkers are proteins. In one embodiment, CD22 and the other biomarkers are nucleic acids.

In one embodiment, the methods of the present invention comprise determining the effective amount of ITGA10 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, ITGA10 and the other biomarkers are proteins. In one embodiment, ITGA10 is nucleic acid.

In one embodiment, the methods of the present invention comprise determining the effective amount of ARRDC4 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, ARRDC4 and the other biomarkers are proteins. In one embodiment, ARRDC4 and the other biomarkers are nucleic acids.

In one embodiment, the methods of the present invention comprise determining the effective amount of C1S (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, C1S and the other biomarkers are proteins. In one embodiment, C1S and the other biomarkers are nucleic acids.

In one embodiment, the methods of the present invention comprise determining the effective amount of PLEKHM1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, PLEKHM1 and the other biomarkers are proteins. In one embodiment, PLEKHM1 and the other biomarkers are nucleic acids.

In one embodiment, the methods of the present invention comprise determining the effective amount of ATP6V1D (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, ATP6V1D and the other biomarkers are proteins. In one embodiment, ATP6V1D and the other biomarkers are nucleic acids.

In one embodiment, the methods of the present invention comprise determining the effective amount of TMED6 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, TMED6 and the other biomarkers are proteins. In one embodiment, TMED6 and the other biomarkers are nucleic acids.

In one embodiment, the methods of the present invention comprise determining the effective amount of FAM135A (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, FAM135A and the other biomarkers are proteins. In one embodiment, FAM135A and the other biomarkers are nucleic acids.

In one embodiment, the methods of the present invention comprise determining the effective amount of CD79A (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CD79A and the other biomarkers are proteins. In one embodiment, CD79A and the other biomarkers are nucleic acids.

In one embodiment, the methods of the present invention comprise determining the effective amount of IL31RA (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, IL31RA and the other biomarkers are proteins. In one embodiment, IL31RA and the other biomarkers are nucleic acids.

In one embodiment, the methods of the present invention comprise determining the effective amount of RHBDF2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, RHBDF2 and the other biomarkers are proteins. In one embodiment, RHBDF2 and the other biomarkers are nucleic acids.

In one embodiment, the methods of the present invention comprise determining the effective amount of HSPG2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, HSPG2 and the other biomarkers are proteins. In one embodiment, HSPG2 and the other biomarkers are nucleic acids.

In one embodiment, the methods of the present invention comprise determining the effective amount of LDLRAD1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, LDLRAD1 and the other biomarkers are proteins. In one embodiment, LDLRAD1 and the other biomarkers are nucleic acids.

In one embodiment, the methods of the present invention comprise determining the effective amount of AAK1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, AAK1 and the other biomarkers are proteins. In one embodiment, v and the other biomarkers are nucleic acids.

In one embodiment, the methods of the present invention comprise determining the effective amount of CD58 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CD58 and the other biomarkers are proteins. In one embodiment, CD58 and the other biomarkers are nucleic acids.

In one embodiment, the methods of the present invention comprise determining the effective amount of CSF1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2 In one embodiment, CSF1 and the other biomarkers are proteins. In one embodiment, CSF1 and the other biomarkers are nucleic acids.

In one embodiment, the methods of the present invention comprise determining the effective amount of KLF6 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, KLF6 and the other biomarkers are proteins. In one embodiment, KLF6 and the other biomarkers are nucleic acids.

In one embodiment, the methods of the present invention comprise determining the effective amount of LAMA3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, LAMA3 and the other biomarkers are proteins. In one embodiment, LAMA3 and the other biomarkers are nucleic acids.

In one embodiment, the methods of the present invention comprise determining the effective amount of TMEM104 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, TMEM104 and the other biomarkers are proteins. In one embodiment, TMEM104 and the other biomarkers are nucleic acids.

In one embodiment, the methods of the present invention comprise determining the effective amount of SPARC (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, SPARC and the other biomarkers are proteins. In one embodiment, SPARC and the other biomarkers are nucleic acids.

In one embodiment, the methods of the present invention comprise determining the effective amount of PVRL4 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, PVRL4 and the other biomarkers are proteins. In one embodiment, PVRL4 and the other biomarkers are nucleic acids.

In one embodiment, the methods of the present invention comprise determining the effective amount of LAD1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, LAD1 and the other biomarkers are proteins. In one embodiment, LAD1 and the other biomarkers are nucleic acids.

In one embodiment, the methods of the present invention comprise determining the effective amount of GLT25D2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, GLT25D2 and the other biomarkers are proteins. In one embodiment, GLT25D2 and the other biomarkers are nucleic acids.

In one embodiment, the methods of the present invention comprise determining the effective amount of TMEM9B (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, TMEM9B and the other biomarkers are proteins. In one embodiment, TMEM9B and the other biomarkers are nucleic acids.

In one embodiment, the methods of the present invention comprise determining the effective amount of CD59 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CD59 and the other biomarkers are proteins. In one embodiment, CD59 and the other biomarkers are nucleic acids.

In one embodiment, the methods of the present invention comprise determining the effective amount of CAPRIN2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CAPRIN2 and the other biomarkers are proteins. In one embodiment, CAPRIN2 and the other biomarkers are nucleic acids.

In one embodiment, the methods of the present invention comprise determining the effective amount of FAM46A (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, FAM46A and the other biomarkers are proteins. In one embodiment, FAM46A and the other biomarkers are nucleic acids.

In one embodiment, the methods of the present invention comprise determining the effective amount of ASB2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, ASB2 and the other biomarkers are proteins. In one embodiment, ASB2 and the other biomarkers are nucleic acids.

In one embodiment, the methods of the present invention comprise determining the effective amount of THBS1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, THBS1 and the other biomarkers are proteins. In one embodiment, THBS1 and the other biomarkers are nucleic acids.

In one embodiment, the methods of the present invention comprise determining the effective amount of CYR61 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CYR61 and the other biomarkers are proteins. In one embodiment, CYR61 and the other biomarkers are nucleic acids.

In one embodiment, the methods of the present invention comprise determining the effective amount of THBS3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, THBS3 and the other biomarkers are proteins. In one embodiment, THBS3 and the other biomarkers are nucleic acids.

In one embodiment, the methods of the present invention comprise determining the effective amount of PDGF (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, PDGF and the other biomarkers are proteins. In one embodiment, PDGF and the other biomarkers are nucleic acids.

In one embodiment, the methods of the present invention comprise determining the effective amount of TGFB1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, TGFB1 and the other biomarkers are proteins. In one embodiment, TGFB12 and the other biomarkers are nucleic acids.

In one embodiment, the methods of the present invention comprise determining the effective amount of the protein PHLDA3 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, PHLDA3 and the other biomarkers are proteins. In one embodiment, PHLDA3 and the other biomarkers are nucleic acids.

In one embodiment, the methods of the present invention comprise determining the effective amount of the protein G6PC (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, G6PC and the other biomarkers are proteins. In one embodiment, G6PC and the other biomarkers are nucleic acids.

In one embodiment, the methods of the present invention comprise determining the effective amount of the protein PGM2L1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, PGM2L1 and the other biomarkers are proteins. In one embodiment, PGM2L1 and the other biomarkers are nucleic acids.

In one embodiment, the methods of the present invention comprise determining the effective amount of the protein C14orf105 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, C14orf105 and the other biomarkers are proteins. In one embodiment, C14orf105 and the other biomarkers are nucleic acids.

In one embodiment, the methods of the present invention comprise determining the effective amount of the protein CTSE (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CTSE and the other biomarkers are proteins. In one embodiment, CTSE and the other biomarkers are nucleic acids.

In one embodiment, the methods of the present invention comprise determining the effective amount of the protein FAS (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, FAS and the other biomarkers are proteins. In one embodiment, FAS and the other biomarkers are nucleic acids.

In one embodiment, the methods of the present invention comprise determining the effective amount of the protein PRF1 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, PRF1 and the other biomarkers are proteins. In one embodiment, PRF1 and the other biomarkers are nucleic acids.

In one embodiment, the methods of the present invention comprise determining the effective amount of the protein CAPN2 (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, CAPN2 and the other biomarkers are proteins. In one embodiment, CAPN2 and the other biomarkers are nucleic acids.

In one embodiment, the methods of the present invention comprise determining the effective amount of the protein HAL (as defined in Tables 1 or 2), and optionally at least 1, at least 2, at least 3, at least 4, at least 5, at least 6, at least 7, at least 8, at least 9, at least 10 or at least 15 of the other biomarkers listed in Tables 1 and 2. In one embodiment, HAL and the other biomarkers are proteins. In one embodiment, HAL and the other biomarkers are nucleic acids.

In a more specific embodiment, the present invention provides methods for diagnosing, prognosing, monitoring or evaluating the risk of fibrosis in a patient, comprising determining an effective amount of SPARC (as defined in Tables 1 or 2) in a sample obtained from a patient. In one embodiment, SPARC is a protein. In one embodiment, SPARC is a nucleic acid.

In a another specific embodiment, the present invention provides methods for diagnosing, prognosing, monitoring or evaluating the risk of diabetes or a prediabetic condition in a patient, comprising determining an effective amount of SPARC (as defined in Tables 1 or 2) in a sample obtained from a patient. In one embodiment, SPARC is a protein. In one embodiment, SPARC is a nucleic acid.

In another specific embodiment, the present invention provides methods for diagnosing, prognosing, monitoring or evaluating the risk of fibrosis in a patient, comprising determining an effective amount of CYR61 (as defined in Tables 1 or 2) in a sample obtained from a patient. In one embodiment, CYR61 is a protein. In one embodiment, CYR61 is a nucleic acid.

In another specific embodiment, the present invention provides methods for diagnosing, prognosing, monitoring or evaluating the risk of diabetes or a prediabetic condition in a patient, comprising determining an effective amount of CYR61 (as defined in Tables 1 or 2) in a sample obtained from a patient. In one embodiment, CYR61 is a protein. In one embodiment, CYR61 is a nucleic acid.

In another specific embodiment, the present invention provides methods for diagnosing, prognosing, monitoring or evaluating the risk of fibrosis in a patient, comprising determining an effective amount of PDGF (as defined in Tables 1 or 2) in a sample obtained from a patient. In one embodiment, PDGF is a protein. In one embodiment, PDGF is a nucleic acid.

In another specific embodiment, the present invention provides methods for diagnosing, prognosing, monitoring or evaluating the risk of diabetes or a prediabetic condition in a patient, comprising determining an effective amount of PDGF (as defined in Tables 1 or 2) in a sample obtained from a patient. In one embodiment, PDGF is a protein. In one embodiment, PDGF is a nucleic acid.

In another specific embodiment, the present invention provides methods for diagnosing, prognosing, monitoring or evaluating the risk of fibrosis in a patient, comprising determining an effective amount of TGFB1 (as defined in Tables 1 or 2) in a sample obtained from a patient. In one embodiment, TGFB1 is a protein. In one embodiment, TGFB1 is a nucleic acid.

In another specific embodiment, the present invention provides methods for diagnosing, prognosing, monitoring or evaluating the risk of diabetes or a prediabetic condition in a patient, comprising determining an effective amount of TGFB1 (as defined in Tables 1 or 2) in a sample obtained from a patient. In one embodiment, TGFB1 is a protein. In one embodiment, TGFB1 is a nucleic acid.

In a more specific embodiment, the present invention provides methods for diagnosing, prognosing, monitoring or evaluating the risk of fibrosis in a patient, comprising determining an effective amount of MVP (as defined in Tables 1 or 2) in a sample obtained from a patient. In one embodiment, MVP is a protein. In one embodiment, MVP is a nucleic acid.

In a another specific embodiment, the present invention provides methods for diagnosing, prognosing, monitoring or evaluating the risk of diabetes or a prediabetic condition in a patient, comprising determining an effective amount of MVP (as defined in Tables 1 or 2) in a sample obtained from a patient. In one embodiment, MVP is a protein. In one embodiment, MVP is a nucleic acid.

In another specific embodiment, the present invention provides methods for diagnosing, prognosing, monitoring or evaluating the risk of fibrosis in a patient, comprising determining an effective amount of PGM2L1 (as defined in Tables 1 or 2) in a sample obtained from a patient. In one embodiment, PGM2L1 is a protein. In one embodiment, PGM2L1 is a nucleic acid.

In a another specific embodiment, the present invention provides methods for diagnosing, prognosing, monitoring or evaluating the risk of diabetes or a prediabetic condition in a patient, comprising determining an effective amount of PGM2L1 (as defined in Tables 1 or 2) in a sample obtained from a patient. In one embodiment, PGM2L1 is a protein. In one embodiment, PGM2L1 is a nucleic acid.

In another specific embodiment, the present invention provides methods for diagnosing, prognosing, monitoring or evaluating the risk of fibrosis in a patient, comprising determining an effective amount of ANXA3 (as defined in Tables 1 or 2) in a sample obtained from a patient. In one embodiment, ANXA3 is a protein. In one embodiment, ANXA3 is a nucleic acid.

In a another specific embodiment, the present invention provides methods for diagnosing, prognosing, monitoring or evaluating the risk of diabetes or a prediabetic condition in a patient, comprising determining an effective amount of ANXA3 (as defined in Tables 1 or 2) in a sample obtained from a patient. In one embodiment, ANXA3 is a protein. In one embodiment, ANXA3 is a nucleic acid.

In another specific embodiment, the present invention provides methods for diagnosing, prognosing, monitoring or evaluating the risk of fibrosis in a patient, comprising determining an effective amount of MYOF (as defined in Tables 1 or 2) in a sample obtained from a patient. In one embodiment, MYOF is a protein. In one embodiment, MYOF is a nucleic acid.

In a another specific embodiment, the present invention provides methods for diagnosing, prognosing, monitoring or evaluating the risk of diabetes or a prediabetic condition in a patient, comprising determining an effective amount of MYOF (as defined in Tables 1 or 2) in a sample obtained from a patient. In one embodiment, MYOF is a protein. In one embodiment, MYOF is a nucleic acid.

In another specific embodiment, the present invention provides methods for diagnosing, prognosing, monitoring or evaluating the risk of fibrosis in a patient, comprising determining an effective amount of LYVE1 (as defined in Tables 1 or 2) in a sample obtained from a patient. In one embodiment, LYVE1 is a protein. In one embodiment, LYVE1 is a nucleic acid.

In a another specific embodiment, the present invention provides methods for diagnosing, prognosing, monitoring or evaluating the risk of diabetes or a prediabetic condition in a patient, comprising determining an effective amount of LYVE1 (as defined in Tables 1 or 2) in a sample obtained from a patient. In one embodiment, LYVE1 is a protein. In one embodiment, LYVE1 is a nucleic acid.

In another specific embodiment, the present invention provides methods for diagnosing, prognosing, monitoring or evaluating the risk of fibrosis in a patient, comprising determining an effective amount of SERPINB8 (as defined in Tables 1 or 2) in a sample obtained from a patient. In one embodiment, SERPINB8 is a protein. In one embodiment, SERPINB8 is a nucleic acid.

In a another specific embodiment, the present invention provides methods for diagnosing, prognosing, monitoring or evaluating the risk of diabetes or a prediabetic condition in a patient, comprising determining an effective amount of SERPINB8 (as defined in Tables 1 or 2) in a sample obtained from a patient. In one embodiment, SERPINB8 is a protein. In one embodiment, SERPINB8 is a nucleic acid.

In another specific embodiment, the present invention provides methods for diagnosing, prognosing, monitoring or evaluating the risk of fibrosis in a patient, comprising determining an effective amount of IL18 (as defined in Tables 1 or 2) in a sample obtained from a patient. In one embodiment, IL18 is a protein. In one embodiment, IL18 is a nucleic acid.

In a another specific embodiment, the present invention provides methods for diagnosing, prognosing, monitoring or evaluating the risk of diabetes or a prediabetic condition in a patient, comprising determining an effective amount of IL18 (as defined in Tables 1 or 2) in a sample obtained from a patient. In one embodiment, IL18 is a protein. In one embodiment, IL18 is a nucleic acid.

In another specific embodiment, the present invention provides methods for diagnosing, prognosing, monitoring or evaluating the risk of fibrosis in a patient, comprising determining an effective amount of MRGPRX4 (as defined in Tables 1 or 2) in a sample obtained from a patient. In one embodiment MRGPRX4 is a protein. In one embodiment, MRGPRX4 is a nucleic acid.

In a another specific embodiment, the present invention provides methods for diagnosing, prognosing, monitoring or evaluating the risk of diabetes or a prediabetic condition in a patient, comprising determining an effective amount of MRGPRX4 (as defined in Tables 1 or 2) in a sample obtained from a patient. In one embodiment, MRGPRX4 is a protein. In one embodiment, MRGPRX4 is a nucleic acid.

In another specific embodiment, the present invention provides methods for diagnosing, prognosing, monitoring or evaluating the risk of fibrosis in a patient, comprising determining an effective amount of G6PC (as defined in Tables 1 or 2) in a sample obtained from a patient. In one embodiment G6PC is a protein. In one embodiment, G6PC is a nucleic acid.

In a another specific embodiment, the present invention provides methods for diagnosing, prognosing, monitoring or evaluating the risk of diabetes or a prediabetic condition in a patient, comprising determining an effective amount of G6PC (as defined in Tables 1 or 2) in a sample obtained from a patient. In one embodiment, G6PC is a protein. In one embodiment, G6PC is a nucleic acid.

In another specific embodiment, the present invention provides methods for diagnosing, prognosing, monitoring or evaluating the risk of fibrosis in a patient, comprising determining an effective amount of DDIT4 (as defined in Tables 1 or 2) in a sample obtained from a patient. In one embodiment DDIT4 is a protein. In one embodiment, DDIT4 is a nucleic acid.

In a another specific embodiment, the present invention provides methods for diagnosing, prognosing, monitoring or evaluating the risk of diabetes or a prediabetic condition in a patient, comprising determining an effective amount of DDIT4 (as defined in Tables 1 or 2) in a sample obtained from a patient. In one embodiment, DDIT4 is a protein. In one embodiment, DDIT4 is a nucleic acid.

In another specific embodiment, the present invention provides methods for diagnosing, prognosing, monitoring or evaluating the risk of fibrosis in a patient, comprising determining an effective amount of CSF1 (as defined in Tables 1 or 2) in a sample obtained from a patient. In one embodiment CSF1 is a protein. In one embodiment, CSF1 is a nucleic acid.

In a another specific embodiment, the present invention provides methods for diagnosing, prognosing, monitoring or evaluating the risk of diabetes or a prediabetic condition in a patient, comprising determining an effective amount of CSF1 (as defined in Tables 1 or 2) in a sample obtained from a patient. In one embodiment, CSF1 is a protein. In one embodiment, CSF1 is a nucleic acid.

In another specific embodiment, the present invention provides methods for diagnosing, prognosing, monitoring or evaluating the risk of fibrosis in a patient, comprising determining an effective amount of C14orf105 (as defined in Tables 1 or 2) in a sample obtained from a patient. In one embodiment C14orf105 is a protein. In one embodiment, C14orf105 is a nucleic acid.

In a another specific embodiment, the present invention provides methods for diagnosing, prognosing, monitoring or evaluating the risk of diabetes or a prediabetic condition in a patient, comprising determining an effective amount of C14orf105 (as defined in Tables 1 or 2) in a sample obtained from a patient. In one embodiment, C14orf105 is a protein. In one embodiment, C14orf105 is a nucleic acid.

In another specific embodiment, the present invention provides methods for diagnosing, prognosing, monitoring or evaluating the risk of fibrosis in a patient, comprising determining an effective amount of CD109 (as defined in Tables 1 or 2) in a sample obtained from a patient. In one embodiment CD109 is a protein. In one embodiment, CD109 is a nucleic acid.

In a another specific embodiment, the present invention provides methods for diagnosing, prognosing, monitoring or evaluating the risk of diabetes or a prediabetic condition in a patient, comprising determining an effective amount of CD109 (as defined in Tables 1 or 2) in a sample obtained from a patient. In one embodiment, CD109 is a protein. In one embodiment, CD109 is a nucleic acid.

In another specific embodiment, the present invention provides methods for diagnosing, prognosing, monitoring or evaluating the risk of fibrosis in a patient, comprising determining an effective amount of CTSE (as defined in Tables 1 or 2) in a sample obtained from a patient. In one embodiment CTSE is a protein. In one embodiment, CTSE is a nucleic acid.

In a another specific embodiment, the present invention provides methods for diagnosing, prognosing, monitoring or evaluating the risk of diabetes or a prediabetic condition in a patient, comprising determining an effective amount of CTSE (as defined in Tables 1 or 2) in a sample obtained from a patient. In one embodiment, CTSE is a protein. In one embodiment, CTSE is a nucleic acid.

In another specific embodiment, the present invention provides methods for diagnosing, prognosing, monitoring or evaluating the risk of fibrosis in a patient, comprising determining an effective amount of F2RL1 (as defined in Tables 1 or 2) in a sample obtained from a patient. In one embodiment F2RL1 is a protein. In one embodiment, F2RL1 is a nucleic acid.

In a another specific embodiment, the present invention provides methods for diagnosing, prognosing, monitoring or evaluating the risk of diabetes or a prediabetic condition in a patient, comprising determining an effective amount of F2RL1 (as defined in Tables 1 or 2) in a sample obtained from a patient. In one embodiment, F2RL1 is a protein. In one embodiment, F2RL1 is a nucleic acid.

In another specific embodiment, the present invention provides methods for diagnosing, prognosing, monitoring or evaluating the risk of fibrosis in a patient, comprising determining an effective amount of FAS (as defined in Tables 1 or 2) in a sample obtained from a patient. In one embodiment FAS is a protein. In one embodiment, FAS is a nucleic acid.

In a another specific embodiment, the present invention provides methods for diagnosing, prognosing, monitoring or evaluating the risk of diabetes or a prediabetic condition in a patient, comprising determining an effective amount of FAS (as defined in Tables 1 or 2) in a sample obtained from a patient. In one embodiment, FAS is a protein. In one embodiment, FAS is a nucleic acid.

In another specific embodiment, the present invention provides methods for diagnosing, prognosing, monitoring or evaluating the risk of fibrosis in a patient, comprising determining an effective amount of LTBP1 (as defined in Tables 1 or 2) in a sample obtained from a patient. In one embodiment LTBP1 is a protein. In one embodiment, LTBP1 is a nucleic acid.

In a another specific embodiment, the present invention provides methods for diagnosing, prognosing, monitoring or evaluating the risk of diabetes or a prediabetic condition in a patient, comprising determining an effective amount of LTBP1 (as defined in Tables 1 or 2) in a sample obtained from a patient. In one embodiment, LTBP1 is a protein. In one embodiment, LTBP1 is a nucleic acid.

In another specific embodiment, the present invention provides methods for diagnosing, prognosing, monitoring or evaluating the risk of fibrosis in a patient, comprising determining an effective amount of KLRC3 (as defined in Tables 1 or 2) in a sample obtained from a patient. In one embodiment KLRC3 is a protein. In one embodiment, KLRC3 is a nucleic acid.

In a another specific embodiment, the present invention provides methods for diagnosing, prognosing, monitoring or evaluating the risk of diabetes or a prediabetic condition in a patient, comprising determining an effective amount of KLRC3 (as defined in Tables 1 or 2) in a sample obtained from a patient. In one embodiment, KLRC3 is a protein. In one embodiment, KLRC3 is a nucleic acid.

In another specific embodiment, the present invention provides methods for diagnosing, prognosing, monitoring or evaluating the risk of fibrosis in a patient, comprising determining an effective amount of MR1 (as defined in Tables 1 or 2) in a sample obtained from a patient. In one embodiment MR1 is a protein. In one embodiment, MR1 is a nucleic acid.

In a another specific embodiment, the present invention provides methods for diagnosing, prognosing, monitoring or evaluating the risk of diabetes or a prediabetic condition in a patient, comprising determining an effective amount of MR1 (as defined in Tables 1 or 2) in a sample obtained from a patient. In one embodiment, MR1 is a protein. In one embodiment, MR1 is a nucleic acid.

In another specific embodiment, the present invention provides methods for diagnosing, prognosing, monitoring or evaluating the risk of fibrosis in a patient, comprising determining an effective amount of SUSD2 (as defined in Tables 1 or 2) in a sample obtained from a patient. In one embodiment SUSD2 is a protein. In one embodiment, SUSD2 is a nucleic acid.

In a another specific embodiment, the present invention provides methods for diagnosing, prognosing, monitoring or evaluating the risk of diabetes or a prediabetic condition in a patient, comprising determining an effective amount of SUSD2 (as defined in Tables 1 or 2) in a sample obtained from a patient. In one embodiment, SUSD2 is a protein. In one embodiment, SUSD2 is a nucleic acid.

In another specific embodiment, the present invention provides methods for diagnosing, prognosing, monitoring or evaluating the risk of fibrosis in a patient, comprising determining an effective amount of TRIM22 (as defined in Tables 1 or 2) in a sample obtained from a patient. In one embodiment TRIM22 is a protein. In one embodiment, TRIM22 is a nucleic acid.

In a another specific embodiment, the present invention provides methods for diagnosing, prognosing, monitoring or evaluating the risk of diabetes or a prediabetic condition in a patient, comprising determining an effective amount of TRIM22 (as defined in Tables 1 or 2) in a sample obtained from a patient. In one embodiment, TRIM22 is a protein. In one embodiment, TRIM22 is a nucleic acid.

In another specific embodiment, the present invention provides methods for diagnosing, prognosing, monitoring or evaluating the risk of fibrosis in a patient, comprising determining an effective amount of PRF1 (as defined in Tables 1 or 2) in a sample obtained from a patient. In one embodiment PRF1 is a protein. In one embodiment, PRF12 is a nucleic acid.

In a another specific embodiment, the present invention provides methods for diagnosing, prognosing, monitoring or evaluating the risk of diabetes or a prediabetic condition in a patient, comprising determining an effective amount of PRF1 (as defined in Tables 1 or 2) in a sample obtained from a patient. In one embodiment, PRF1 is a protein. In one embodiment, PRF1 is a nucleic acid.

In another specific embodiment, the present invention provides methods for diagnosing, prognosing, monitoring or evaluating the risk of fibrosis in a patient, comprising determining an effective amount of C7 (as defined in Tables 1 or 2) in a sample obtained from a patient. In one embodiment C7 is a protein. In one embodiment, C7 is a nucleic acid.

In a another specific embodiment, the present invention provides methods for diagnosing, prognosing, monitoring or evaluating the risk of diabetes or a prediabetic condition in a patient, comprising determining an effective amount of C7 (as defined in Tables 1 or 2) in a sample obtained from a patient. In one embodiment, C7 is a protein. In one embodiment C7 is a nucleic acid.

In another specific embodiment, the present invention provides methods for diagnosing, prognosing, monitoring or evaluating the risk of fibrosis in a patient, comprising determining an effective amount of CAPN2 (as defined in Tables 1 or 2) in a sample obtained from a patient. In one embodiment CAPN2 is a protein. In one embodiment, CAPN2 is a nucleic acid.

In a another specific embodiment, the present invention provides methods for diagnosing, prognosing, monitoring or evaluating the risk of diabetes or a prediabetic condition in a patient, comprising determining an effective amount of CAPN2 (as defined in Tables 1 or 2) in a sample obtained from a patient. In one embodiment, CAPN2 is a protein. In one embodiment CAPN2 is a nucleic acid.

In another specific embodiment, the present invention provides methods for diagnosing, prognosing, monitoring or evaluating the risk of fibrosis in a patient, comprising determining an effective amount of NRP1 (as defined in Tables 1 or 2) in a sample obtained from a patient. In one embodiment NRP1 is a protein. In one embodiment, NRP1 is a nucleic acid.

In a another specific embodiment, the present invention provides methods for diagnosing, prognosing, monitoring or evaluating the risk of diabetes or a prediabetic condition in a patient, comprising determining an effective amount of NRP1 (as defined in Tables 1 or 2) in a sample obtained from a patient. In one embodiment, NRP1 is a protein. In one embodiment NRP1 is a nucleic acid.

In another specific embodiment, the present invention provides methods for diagnosing, prognosing, monitoring or evaluating the risk of fibrosis in a patient, comprising determining an effective amount of QPCT (as defined in Tables 1 or 2) in a sample obtained from a patient. In one embodiment QPCT is a protein. In one embodiment, QPCT is a nucleic acid.

In a another specific embodiment, the present invention provides methods for diagnosing, prognosing, monitoring or evaluating the risk of diabetes or a prediabetic condition in a patient, comprising determining an effective amount of QPCT (as defined in Tables 1 or 2) in a sample obtained from a patient. In one embodiment, QPCT is a protein. In one embodiment QPCT is a nucleic acid.

In another specific embodiment, the present invention provides methods for diagnosing, prognosing, monitoring or evaluating the risk of fibrosis in a patient, comprising determining an effective amount of SRPX2 (as defined in Tables 1 or 2) in a sample obtained from a patient. In one embodiment SRPX2 is a protein. In one embodiment, SRPX2 is a nucleic acid.

In a another specific embodiment, the present invention provides methods for diagnosing, prognosing, monitoring or evaluating the risk of diabetes or a prediabetic condition in a patient, comprising determining an effective amount of SRPX2 (as defined in Tables 1 or 2) in a sample obtained from a patient. In one embodiment, SRPX2 is a protein. In one embodiment SRPX2 is a nucleic acid.

In another specific embodiment, the present invention provides methods for diagnosing, prognosing, monitoring or evaluating the risk of fibrosis in a patient, comprising determining an effective amount of HAL (as defined in Tables 1 or 2) in a sample obtained from a patient. In one embodiment HAL is a protein. In one embodiment, HAL is a nucleic acid.

In a another specific embodiment, the present invention provides methods for diagnosing, prognosing, monitoring or evaluating the risk of diabetes or a prediabetic condition in a patient, comprising determining an effective amount of HAL (as defined in Tables 1 or 2) in a sample obtained from a patient. In one embodiment, HAL is a protein. In one embodiment HAL is a nucleic acid.

### LIST OF FIGURES

FIG. 1.

Association of hepatocyte p21 expression with fibrosis stage and laboratory Indices in patients with alcohol-related liver disease (ALD). Hepatocyte p21 expression demonstrated an association with fibrosis stage (1a) in the first cohort; the proportion of hepatocytes that expressed p21 increased with increasing fibrosis stage. Areas of increased α-SMA expression (a marker of activated hepatic stellate cells) were associated with higher hepatocyte p21 expression (1b) than those areas with less α-SMA expression, which were associated with lower hepatocyte p21 expression (1c) even within the same tissue.

FIG. 2.

Quantification of plasma protein levels by ELISA for 12 candidate genes: (a) MVP; (b) PGM2L1; (c) SPARC; (d) CYR61; (e) LYVE1; (f) SERPINB8; (g) ANXA3; (h) MYOF; (i) IL18; (j) MRGPRX4; (k) G6PC and (l) CSF1 in samples derived from patients with fibrosis or established cirrhosis, as compared to healthy controls. In addition, hexagonal symbols at the top of each plot indicate the protein level in whole liver tissue samples harvested at liver transplantation (three filled symbols indicate that a high level of protein was detected, one filled symbol indicates that protein was detected in liver tissue but at lower levels, and a single non-filled symbol indicates that the protein was only detected in one liver sample (with very severe disease)).

### EXAMPLES

### Example 1

### Materials and Methods

Liver sections from two distinct cohorts were studied: an ascertainment cohort (n=42, patients with the full spectrum ALD) and a validation cohort, to confirm the outcome findings in the ascertainment cohort, (n=77, patients with ALD-related cirrhosis). Patients were reviewed at least every 6 months until death, an adverse liver-related outcome or the censor point. Immunochemistry was used to study the cell cycle phase markers.

### Results

In this study, p21 expression was used as a measure of senescence in hepatocytes.

### Hepatocyte Senescence and Fibrosis—First and Second Cohorts

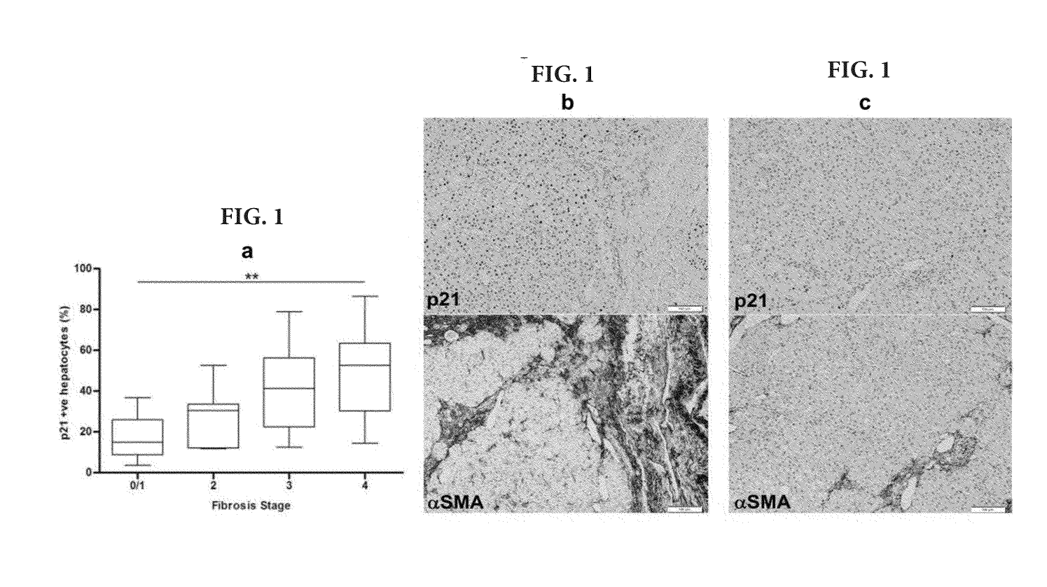

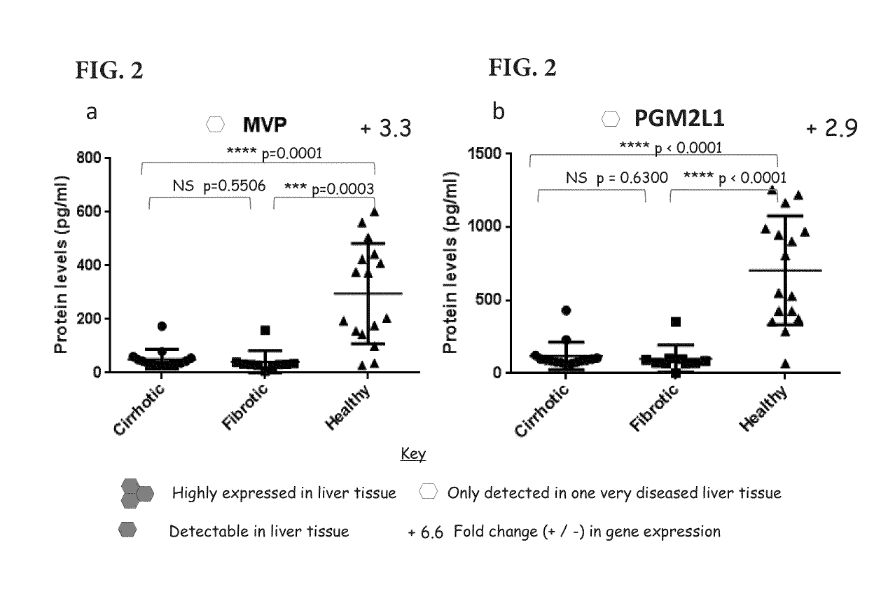

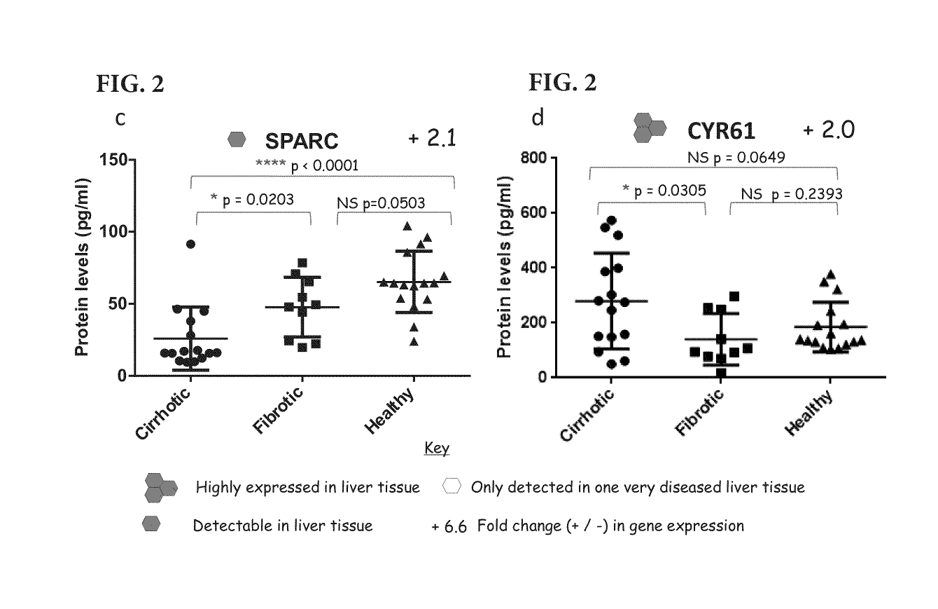

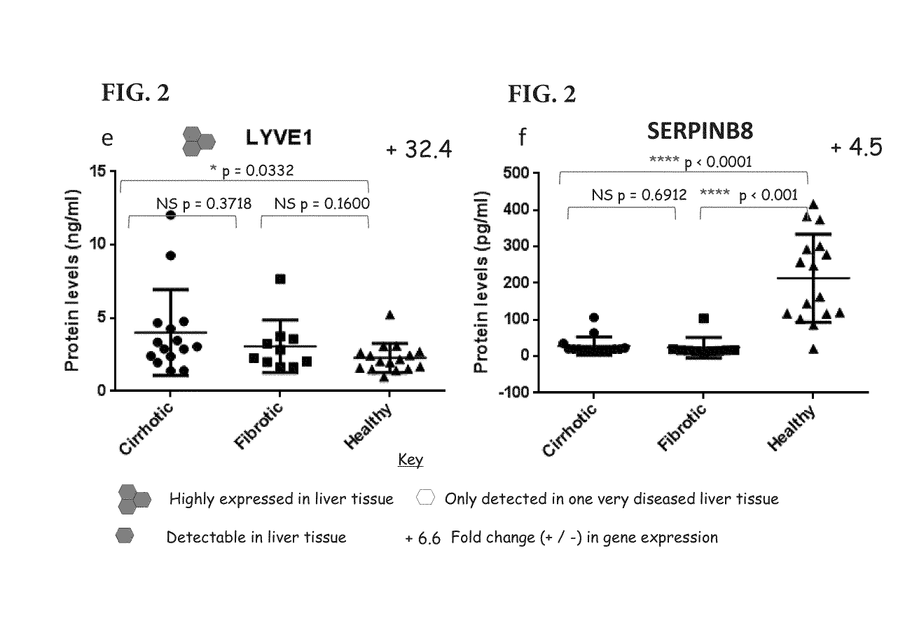

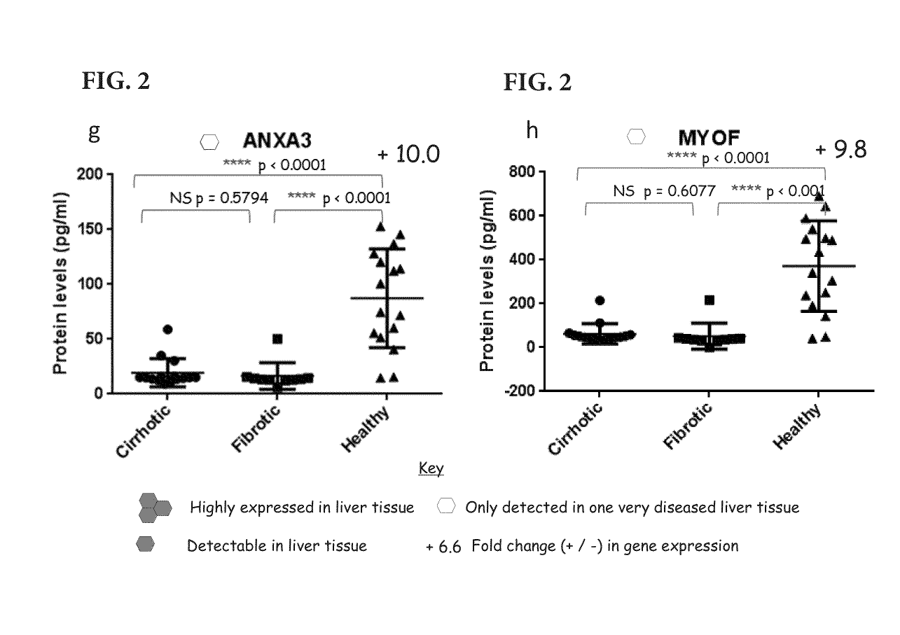

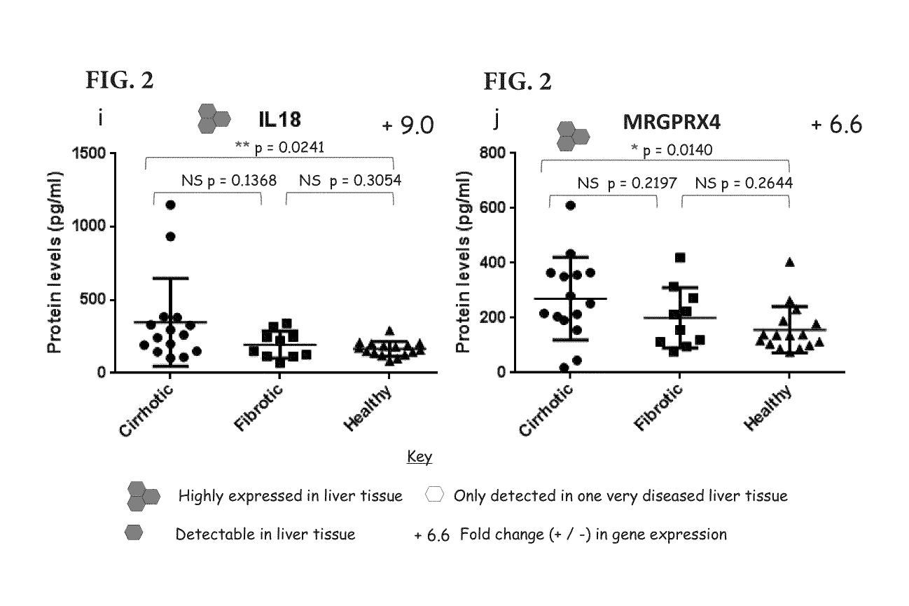

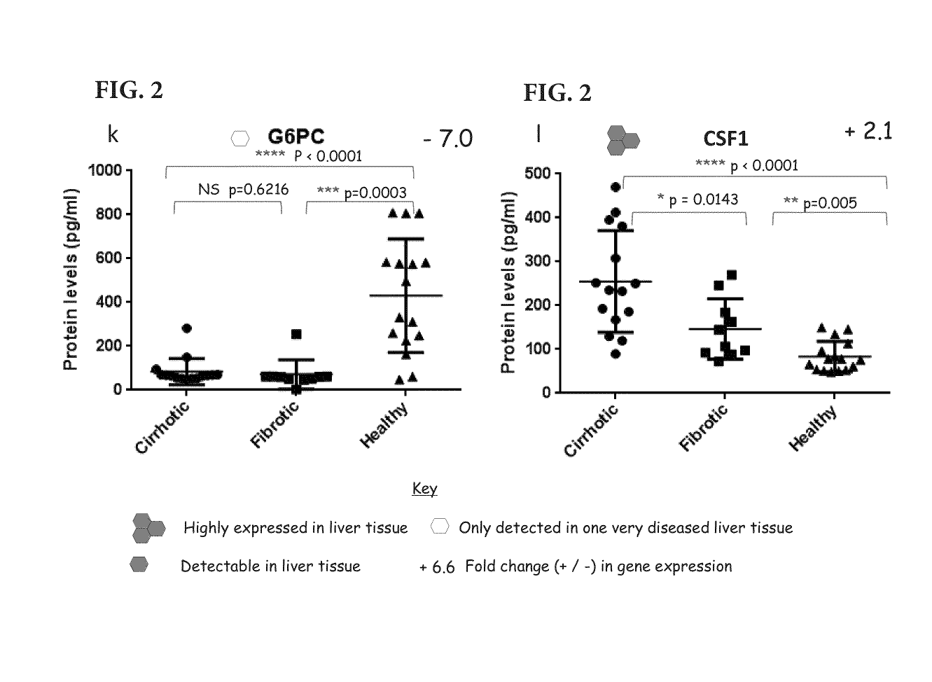

In the first cohort, there was a positive correlation between hepatocyte p21 expression and fibrosis stage (p=0.002; FIG. 1a), which remained significant (p=0.005) after multivariate analysis when controlled for age, sex, presence of alcohol-related steatohepatitis and MELD score. A similar analysis could not be undertaken in the second cohort as all patients had cirrhosis. Areas of liver with increased hepatic stellate cell activation (the major cell type involved in liver fibrosis) were associated with higher hepatocyte p21 expression; in contrast, areas of liver with fewer activated hepatic stellate cells were associated with lower hepatocyte p21 expression, even within the same tissue (FIGS. 1b & 1c). Expression of p21 was diffuse and not restricted to periportal or perivenular zones.

### Hepatocyte Senescence and Outcome—First and Second Cohorts

Analysis with the Log-rank test (univariate analysis) in the first cohort revealed that patients with higher hepatocyte p21 expression (above the median) were more likely to develop an adverse liver-related outcome than those with lower p21 expression (p=0.03).

### Discussion

There was a progressive increase in hepatocyte senescence with increasing fibrosis stage in ALD. This indicates that senescent hepatocytes accumulate with progressive fibrosis. The geographic association between hepatocyte p21 expression and hepatic stellate cell activation (shown by α-SMA expression) further indicates that hepatocyte senescence, activation of hepatic stellate cells and fibrosis are linked.

In conclusion, this study demonstrated that impaired cell cycle progression in hepatocytes, hepatocyte senescence and progressive fibrosis in ALD were inter-related. Further, there was a strong relation between the proportion of senescent hepatocytes and an adverse liver-related outcome.

### Example 2

This study investigated the insulin pathway in senescent hepatocytes.

Experiments were performed using (a) normally growing, serum-starved HepG2 hepatocytes (b) normally growing, serum-starved and insulin-stimulated HepG2 hepatocytes (c) H₂O₂-induced senescent, serum-starved HepG2 hepatocytes and (d) H₂O₂-induced senescent, serum-starved and insulin-stimulated HepG2 hepatocytes.

### PI3K-Akt Pathway

Insulin stimulation led to phosphorylation and activation of Akt, in both normal and senescent HepG2 hepatocytes.

Phosphorylation and activation of Akt is signalled through four important downstream pathways.

1. Phosphorylation of S6 kinase—phosphorylation of S6 kinase was intact in both normal and senescent HepG2 hepatocytes. This indicates that phosphorylation and inhibition of TSC2 resulting in activation of mTORC1 is intact in both conditions.

2. Phosphorylation of GSK—phosphorylation of GSK was also intact in normal and senescent HepG2 hepatocytes following activation of Akt.

3. Phosphorylation of AS160—AS160 was phosphorylated in normal hepatocytes but not in senescent HepG2 hepatocytes.

4. Phosphorylation of FoxO1 at serine 256—FoxO1 was unphosphorylated in senescent HepG2 hepatocytes. Further FoxO1 remained within the HepG2 hepatocyte nucleus despite insulin stimulation.

Thus, this indicates that at least two of the four downstream effector pathways of Akt were blemished in HepG2 hepatocyte senescence.

### Ras-MAPK Pathway

Insulin stimulation resulted in phosphorylation of Erk1/2 (p44/42 MAPK) in normal and senescent HepG2 hepatocytes. This indicates that the Ras-MAPK cascade is activated upon insulin stimulation in both circumstances.

### Discussion

In senescent hepatocytes, in the crucial PI3K-Akt pathway that mediates the metabolic effects of insulin, there were defects in phosphorylation of mediators in at least two of the four downstream pathways of Akt. Inhibition of AS160 through phosphorylation is a vital step in the translocation of glucose transporter (GLUT4) from cytoplasm to plasma membrane and therefore regulates cellular glucose uptake. The lack of phosphorylation of AS160 with insulin stimulation in senescent HepG2 hepatocytes indicates that this process is likely to be affected during senescence.

In normal hepatocytes, GLUT2 is the major glucose transporter and unlike GLUT4, it is not regulated by insulin. To this end, quantitative PCR analysis of normal and senescent HepG2 hepatocytes showed down regulation of GLUT2 and up regulation of GLUT4 in senescent HepG2 hepatocytes. Thus it is plausible that the non-phosphorylation of AS160 with insulin stimulation will have an impact on glucose uptake of hepatocytes, especially during senescence.

In normal hepatocytes, insulin stimulation leads to phosphorylation and nuclear exclusion of FoxO transcription factor which otherwise remains within the nucleus and promotes expression of genes involved in cell cycle arrest, detoxification of reactive oxygen species, DNA repair and gluconeogenesis. In this study, in senescent HepG2 hepatocytes, activated Akt failed to phosphorylate FoxO1 and the FoxO1 remained within the nucleus despite insulin stimulation. This constitutive activity of FoxO is likely to induce insulin resistance in senescent hepatocytes.

FoxO transcription factors are also phosphorylated by stress-inducible kinases such as JNK and MST1 and this leads to nuclear localisation and increased transcriptional activity. The activation of FoxO through phosphorylation by JNK and MST1 is dominant to the inhibitory phosphorylation by Akt. Therefore, although FoxO localises to cytoplasm in cells treated with growth factors such as insulin under normal conditions, it remains in the nucleus under stress conditions even in the presence of growth factors, as in this study.

In this study, inhibition of insulin pathway in senescent hepatocytes is not complete—i.e. not all insulin functions are equally affected. Such uncoupled insulin regulation is known as “selective insulin resistance” and has been recognised in liver and adipocytes. Maintenance of senescent state requires transcriptional activity of FoxO of cell cycle inhibitory genes even in the presence of growth factors and it is conceivable that gluconeogenesis is an unwanted but unavoidable consequence of such FoxO activity. On the other hand, the mTORC1/S6 kinase which is involved in protein synthesis, remain sensitive to insulin as senescent cells remain “active” secreting a variety of biologically active factors.

Inactivation of GSK3 through phosphorylation and enhanced glycogenesis and glycogen accumulation is common in senescence. Abnormal accumulation of glycogen in nucleus has also been described in senescent hepatocytes. Although, in this study, the GSK3 was phosphorylated with insulin stimulation in senescent hepatocytes, phospho-GSK3 levels were similar in normal and senescent hepatocytes prior to insulin stimulation.

In conclusion, this study demonstrates selective insulin resistance in senescent hepatocytes explaining the development of insulin resistance in advanced liver disease.

### Example 3

### Introduction

The aim of this study was to characterise the alteration in gene expression of senescent hepatocytes.

### Materials and Methods

### Cell Culture and Induction of Senescence

The human liver cell line HepG2 was grown in Dulbecco's Modified Eagle Medium containing 10% foetal calf serum and standard antibiotics (100 U/ml Penicillin, 100 μg/ml Streptomycin). Hepatocytes were seeded in 6-well plates at a density of 5×105 per well and allowed to adhere overnight before treatment. Senescence was induced by treating the cells for 60 minutes with a final concentration of 0.5 mM H₂O₂ in culture media or, in culture media alone. Hepatocytes were then washed three times with PBS and incubated at 37° C., 5% CO₂ for 5 days before observing cell morphology and testing for senescent phenotype.

### Evaluation of Senescence-Associated β-Galactosidase (SA-β-GAL) Activity

SA-β-GAL activity was evaluated using Senescence β-Galactosidase Staining Kit (Cell Signaling Technology™) following the manufacturer's recommendations. Following overnight incubation at 37° C., hepatocytes were examined under a light microscope for development of blue staining.

### Immunofluorescence

The following antibodies were used for immunofluorescence assays: (i) unconjugated rabbit anti-Mcm-2 (Cell Signaling; concentration 1:50, FITC fluorochrome-conjugated donkey anti-rabbit secondary antibody), (ii) Alexa Fluor® 647 conjugated rabbit anti-PH3 (Cell Signaling; concentration 1:50), (iii) Alexa Fluor® 488 conjugated rabbit anti-p21 (Cell Signaling; concentration 1:50), (iv) unconjugated mouse anti-HP1γ (Millipore; concentration 1:500; Cy3 fluorochrome-conjugated donkey anti-mouse secondary antibody) and (v) unconjugated rabbit anti-HMGI-C(Santa Cruz Biotechnology; concentration 1:500; FITC fluorochrome-conjugated donkey anti-rabbit secondary antibody). Cell cycle arrest was determined by immunofluorescence for Mcm-2, PH3 and p21. In brief, cells (mock and H2O2-treated), grown on coverslips, were washed twice with PBS before being fixed with 4% PFA for 15 minutes at room temperature and permeabilised with 0.2% Triton X-100 for 5 minutes. Cells were washed again before incubation at room temperature with specific antibodies, diluted in PBST (0.5% Tween-20 in PBS) containing 1% normal goat serum, for 45 minutes. Where required (Mcm-2), cells were washed with PBST before incubation with a species specific fluorochrome conjugated secondary antibody. DAPI (4′, 6′ diamino-2-phenylindole; concentration 1:500) was used to stain the nucleus in all of these experiments. Coverslips were mounted on glass slides using fluorescence mounting medium (Dako) and visualised by UV-microscopy.

### Determination of SASPs and Secretory Proteins in Conditioned Media

The levels of SASPs (IL-6 and IL-8) and fibrinogen in conditioned media from H₂O₂-treated and untreated HepG2 cells were determined using the ELISA Ready-SET-Go kits (Human IL-6 and Human IL-8; eBioscience) and Fibrinogen Human ELISA Kit (Abcam) according to the manufacturers' recommendations. The levels of α-fetoprotein and retinol-binding protein were measured using time-resolved fluoroimmunoassay (AutoDELFIA™ hAFP) and ‘Top-down’ mass spectrometric analysis, respectively, by the Department of Clinical Biochemistry at Cambridge University Hospitals, Cambridge, UK.

### RNA Extraction and Microarray Analysis

RNA extraction was performed using Qiagen RNeasy Plus Mini Kit with a standard protocol. RNA quality and quantity were measured using spectrophotometry at 260 and 280 nm and on 2100 Bioanalyzer Eukaryote Total RNA Nano Series II chip (Agilent). Microarray experiments were performed by Genomics CoreLab—Cambridge NIHR Biomedical Research Centre, Cambridge, UK. RNA was prepared as described above and processed using Affymetrix GeneChip® Human Gene 1.0 ST arrays. RNA integrity number (RIN) values were between 9.7 and 10 for all samples. The labelling of the sample material, hybridisation and scanning of the microarrays was carried out according to Affymetrix standard protocols. Normalised expression estimates were obtained from the raw intensity values using a probe level linear model pre-processing algorithm available in the Bioconductor library AffyPLM (fitPLM) using default settings. Differential expression of genes between cells treated with H₂O₂ and matched control cells was assessed using an empirical Bayes' statistic and p-values were adjusted for multiple testing using the Bioconductor library limma (eBayes). Genes showing up-regulation or down-regulation during senescence were taken as those showing differential expression (adjusted p-value <0.01) of least 2-fold. Principal component analysis plots were generated using the Bioconductor library affycoretools (plotPCA). Genes were annotated with gene ontology terms using DAVID (http://david.abcc.ncifcrf.gov/). Significant enrichment of gene ontology terms in the up-regulated and down-regulated different expressed gene lists was assessed using FuncAssociate 2.0 (http://llama.mshri.on.ca/funcassociate/) which performs Fisher's exact test analysis with empirical resampling to correct for multiple hypotheses.

### Generation of Differentially Expressed Gene Lists

Initial normalisation of the array data and generation of a differentially expressed gene list was carried out using R (version 12.14.2), using the following packages: limma, affy, affyPLM and affycoretools. Normalised expression estimates from the raw intensity values were carried out using the probe level linear model pre-processing algorithm available in the Bioconductor library AffyPLM (fitPLM) using default settings. To inspect arrays for potential artefacts, intra array quality was visualised using image plots of raw, weights, residuals and the sign of residuals. Sample quality and effect of normalisation was visualised by inspection of plots of sample intensity before and after normalisation, relative log expression (RLE), normalised unscaled standard error (NUSE), RNA degradation, and principal components before and after normalisation. MA plots were also generated comparing chips to a pseudo-median reference chip as well as within sample groups (cell line and treatment status). Differential expression of genes between cells treated with H₂O₂ and matched control cells was assessed using an empirical Bayes' statistic and p-values were adjusted for multiple testing using the Bioconductor library limma. A linear model was fitted (ImFit) and used to compute estimated coefficients and standard errors between groups (contrasts.fit). Empirical Bayes shrinkage of standard errors was then used to rank genes in order of evidence for differential expression (eBayes). Principal component analysis plots were generated using the Bioconductor library affycoretools (plotPCA) to examine the relationship amongst samples from the same cell line and across cell lines. A set of genes showing up-regulation or down-regulation during senescence was taken as those showing differential expression (adjusted p<0.01) of least 2-fold between H₂O₂-treated and untreated samples.

### Functional Annotation of Genes

To obtain functional roles of individual genes, genes present in the senescence gene list were annotated with gene ontology terms using DAVID (http://david.abcc.ncifcrf.gov/). Significant enrichment of gene ontology terms in the up-regulated and down-regulated differently expressed gene lists was assessed using FuncAssociate 2.0 (http://llama.mshri.on.ca/funcassociate/) which performs Fisher's exact test analysis with empirical resampling to correct for multiple hypotheses.

Mapping of Gene Lists onto Pathway Diagrams

In order to visualise dysregulation of pathways, gene lists were used to shade KEGG (http://www.kegg.jp/) pathway diagrams via the KEGG REST API. Genes were shaded according to presence in the up-regulated gene list (red), down-regulated gene list (green), or neither (unshaded, white). Corresponding heatmaps were generated for the pathways showing genes from the pathway with significant differential expression (adjusted p<0.01). Expression was plotted as modified z-score values (using median and median of absolute deviation) and shaded according to high expression (red) and low expression (green).

### Analysis of Publically Available Microarray Data

Publically available microarray data was used in order to confirm the link between hepatocyte senescence and chronic liver disease, and examine the ability of this hepatocyte senescence microarray signature to classify patients by disease status. Datasets contained samples of diseased and normal liver tissue. Diseased liver tissue was representative of steatohepatitis, n=12 vs. 13 normal (GSE33814), alcoholic hepatitis, n=15 vs. 7 normal (GSE28619), and HCV-related cirrhosis, n=41 vs. 19 normal (GSE14323). Normalised array data was downloaded from the Gene Expression Omnibus (http://www.ncbi.nlm.nih.gov/geo/) and processed using R (version 12.14.2). Genes on the microarray were subset to the set of genes showing up-regulation or down-regulation during senescence and expression displayed as a heatmap (heatmap.2 from the gplots package, using row scaling) following unsupervised clustering of samples and genes (hclust). Genes with differential expression between disease and normal were calculated using Empirical Bayes shrinkage of standard (eBayes) prior to subsetting the microarray. Gene-set enrichment analysis was performed to assess enrichment of the genes showing up-regulation and down-regulation in disease state using the GSEA GUI software (http://www.broadinstitute.org/gsea/). Briefly, the analysis was run as a pairwise comparison ranking genes on the signal2noise ratio between diseased and normal samples, 10000 phenotype permutations were used in the p-value calculation.

### Quantitative RT-PCR

Real time one step RT-PCR reactions were carried out on the Rotor-Gene 6000 (Qiagen) using the TaqMan® Fast Virus 1-Step Master Mix system (Life Technologies) in a final volume of 20 μl, using primers (listed in Supplementary Table) and a 5′FAM™ labelled TaqMan® MGB probe (Life Technologies; catalogue number 4331182) following the manufacturer's recommendations. A total of 5 ng of RNA was amplified using the reaction conditions 50° C. for 5 minutes (RT), denaturation at 95° C. for 20 seconds followed by 50 cycles of 95° C. for 3 seconds and 60° C. extension step for 30 seconds. Expression of all genes was normalised to 18S levels.

### Human Liver Samples

Frozen liver tissue (30 mg) from explants of three patients with liver cirrhosis who underwent liver transplantation was obtained for quantitative RT-PCR analysis. Frozen liver tissue (30 mg) from three age-matched patients who underwent partial hepatectomy for liver cysts was obtained (normal controls). These specimens were taken distant to the liver cyst and demonstrated normal histology on H&E-stained sections. RNA extraction and quantitative RT-PCT were performed as mentioned above.

### Results

### Oxidative Stress Induced Senescence in Hepatocytes

Time and dose curves were generated to determine the optimum effect and incubation with 0.5 mM H₂O₂ in culture medium for 60 minutes induced senescence in >95% of HepG2 hepatocytes. Senescence in H₂O₂-treated HepG2 hepatocytes was confirmed using the following:

1. Cellular morphology: H₂O₂-treated hepatocytes revealed the characteristic changes of senescence including enlargement, flattening and elongation when compared to untreated hepatocytes.

2. SA-β-GAL expression: Over 95% of H₂O₂-treated hepatocytes but less than 5% of untreated hepatocytes expressed SA-β-GAL.

3. Cell cycle markers: Mcm-2 (cell cycle entry) was present in 95% of untreated hepatocytes and 74% of H₂O₂-treated hepatocytes. PH3 (M phase) was present in 10% of untreated hepatocytes but none of the H₂O₂-treated hepatocytes. p21 (cell cycle inhibition) was present in none of the untreated hepatocytes but 93% of H₂O₂-treated cells indicating cell cycle arrest in H₂O₂-treated hepatocytes. 4. Expression of cell cycle arrest genes: Gene expression of p53, p21 and p16, major components of the induction and maintenance of senescence were up-regulated significantly in H₂O₂-treated hepatocytes compared to untreated.

5. SAHFs: All hepatocytes treated with H₂O₂ expressed HP1γ and HMGI-C, whereas SAHF were not detected in any untreated hepatocytes.

6. SASPs: The levels of IL-8 were significantly higher in the conditioned media from H₂O₂-treated hepatocytes compared to media from untreated hepatocytes. However, IL-6 was detected at low levels in both H₂O₂-treated and untreated conditioned media.

Characteristic morphological changes, the presence of SA-β-GAL, cell cycle arrest and SAHFs along with high levels of SASPs in conditioned media confirm senescence in H₂O₂-treated HepG2 hepatocytes.

### Differential Expression of Genes in HepG2 Senescence

To identify gene expression changes in hepatocytes during senescence, four samples of senescent HepG2 hepatocytes (H₂O₂-induced) and four samples of ‘normal’ untreated HepG2 cells were studied. There was a clear separation in gene expression between the normal and senescent HepG2 cell line on principal component analysis.

102 genes which were found to have significant differential gene expression in senescent hepatocytes compared to untreated cells are listed in Table 3:

TABLE 3

| Gene | Fold Change |
|---|---|
| LYVE1 | +32.45 |
| AREG | +10.91 |
| CD109 | +10.69 |
| ANXA3 | +10.00 |
| MYOF | +9.79 |
| IL18 | +9.01 |
| LIPH | +8.73 |
| MRGPRX4 | +6.62 |
| VNN1 | +6.56 |
| ANXA1 | +6.19 |
| SERPINE2 | +6.10 |
| SRPX2 | +5.44 |
| QPCT | +5.22 |
| F2RL1 | +5.08 |
| KITLG | +4.64 |
| MR1 | +4.63 |
| LY96 | +4.63 |
| EMP3 | +4.60 |
| LAPTM5 | +4.58 |
| SERPINB8 | +4.53 |
| PLA2G2A | +4.32 |
| CLDN6 | +4.30 |
| ANKRD1 | +4.28 |
| SLC16A4 | +4.08 |
| SUSD2 | +3.96 |
| KLRC3 | +3.76 |
| C7 | +3.68 |
| TRIM22 | +3.60 |
| GLIPR2 | +3.51 |
| SLFN5 | +3.41 |
| NRP1 | +3.38 |
| CRYAB | +3.36 |
| MVP | +3.30 |
| CD9 | +3.17 |
| SPINT1 | +3.13 |
| OR52N4 | +3.10 |
| LTBP1 | +3.09 |
| ITGA3 | +3.05 |
| KLRC2 | +3.04 |
| EMP1 | +2.93 |
| PLAU | +2.87 |
| AXL | +2.83 |
| LGALS1 | +2.83 |
| NAV3 | +2.80 |
| CD3D | +2.75 |
| SAA4 | +2.74 |
| SYT11 | +2.74 |
| CHI3L1 | +2.69 |
| SYTL2 | +2.66 |
| GBA | +2.59 |
| ABCC3 | +2.58 |
| STX3 | +2.58 |
| KLRC4 | +2.57 |
| PTAFR | +2.52 |
| TAX1BP3 | +2.50 |
| TMC7 | +2.50 |
| KLRK1 | +2.48 |
| KIAA1199 | +2.41 |
| SPOCK2 | +2.39 |
| CD22 | +2.38 |
| ITGA10 | +2.38 |
| ARRDC4 | +2.35 |
| C1S | +2.33 |
| PLEKHM1 | +2.30 |
| ATP6V1D | +2.22 |
| TMED6 | +2.22 |
| FAM135A | +2.21 |
| CD79A | +2.20 |
| IL31RA | +2.20 |
| RHBDF2 | +2.19 |
| HSPG2 | +2.17 |
| LDLRAD1 | +2.16 |
| AAK1 | +2.16 |
| CD58 | +2.14 |
| CSF1 | +2.14 |
| KLF6 | +2.13 |
| LAMA3 | +2.11 |
| TMEM104 | +2.08 |
| SPARC | +2.07 |
| PVRL4 | +2.06 |
| LAD1 | +2.05 |
| GLT25D2 | +2.04 |
| TMEM9B | +2.04 |
| CD59 | +2.03 |
| CAPRIN2 | +2.03 |
| FAM46A | +2.02 |
| ASB2 | +2.01 |
| THBS1 | +2.01 |
| CYR61 | +2.00 |
| THBS3 | +2.00 |
| PDGF | +2.00 |
| TGFB1 | +2.00 |
| PHLDA3 | +2.00 |
| DDIT4 | −2.10 |
| G6PC | −7.00 |
| PGM2L1 | +2.90 |
| C14orf105 | +6.20 |
| CTSE | +8.60 |
| FAS | +3.60 |
| PRF1 | +3.30 |
| CAPN | +7.20 |
| HAL | −5.50 |

### Hepatocyte Senescence and Fibrosis

This study also demonstrated a potential link between senescent hepatocytes and stellate cell activation. PDGF, the most potent mitogen described thus far and a powerful chemoattractant of stellate cells was up-regulated in hepatocyte senescence. TGFβ1, a soluble signalling factor secreted by senescent cells, in addition to its role as a potent hepatocyte cell cycle inhibitor, is a powerful pro-fibrotic cytokine, and was also up-regulated in hepatocyte senescence. LTBP1 which is essential for the secretion and activation of TGFβs and AREG which causes proliferation of fibroblasts were up-regulated. Thus, it is plausible that these soluble signalling factors secreted by senescent hepatocytes could lead to proliferation and activation of hepatic stellate cells, which may in turn may explain the strong independent association seen between hepatocyte p21 expression and fibrosis stage in chronic liver diseases. This is further corroborated by the geographic association between hepatocyte p21 expression and hepatic stellate cell activation (shown by α-SMA expression) as shown in FIG. 1 (Example 1).

### Association Between Senescence and Metabolism of Glucose, Lipids and Proteins

Expression of genes involved in hepatocyte metabolism of glucose, lipids and proteins and crucial signal transduction pathways such as PI3K/Akt, MAPK, Jak/Stat, NF-κB, TGFβ, IGF1 and insulin were altered in hepatocyte senescence. Analysis of the conditioned media demonstrated reduced hepatocyte synthetic function in senescent hepatocytes. This, and differential expression of genes involved in metabolism and signal transduction, suggest that senescent hepatocytes do not function as normal mature hepatocytes and may explain the relation of hepatocyte senescence with hepatic dysfunction and adverse liver-related outcome seen in chronic liver disease. Hypocholesterolaemia and impaired glucose tolerance seen in advanced stages of chronic liver disease may also be the consequences of altered metabolism of senescent hepatocytes which accumulate in advanced chronic liver disease.

### Example 4

### Introduction

The RNA microarray performed in Example 3 revealed 102 genes exhibiting altered expression in senescent HepG2 cell lines as compared to untreated HepG2 cell lines. This study examined the protein expression of twelve of these genes within samples derived from patients with liver fibrosis and healthy controls.

### Materials and Methods

EDTA plasma samples from a cohort of patients (n=40) with a recent biopsy showing advanced fibrosis or established cirrhosis were studied. The samples were derived from patients with a wide range of underlying aetiology. Comparisons were made with control plasma samples derived from healthy blood donors provided by the Scottish Blood Service (n=20). Plasma protein levels for twelve candidate genes (CYR61, CSF1, IL-18, MRGPRX4, LYVE1, SPARC, MVP, PGM2L1, ANXA3, MYOF, SERPINB8, and G6PC) were measured using commercially available sandwich enzyme-linked immunosorbant assay (ELISA) kits.

Protein levels of these twelve candidate genes were also measured in 5 whole liver tissue explanted samples harvested at liver transplantation.

Data were analysed using SPSS.

### Results

The ELISAs conducted for the twelve candidate genes showed clear differences in protein expression between healthy controls and patients with liver fibrosis (FIGS. 2a-2l). In a smaller number, there was also a distinction between cirrhosis and fibrosis stages short of cirrhosis.

Of the eleven genes that were shown to exhibit increased RNA expression in senescent hepatocytes (SPARC, CYR61, MVP, PGM2L1, ANXA3, MYOF, LYVE1, SERPINB8, 1L18, MRGPRX4, and CSF1), only five exhibited a corresponding increase in protein expression in plasma samples from patients with cirrhosis (CYR61, CSF1, IL-18, MRGPRX4 and LYVE1). Thus, the remaining six genes (SPARC, MVP, PGM2L1, ANXA3, MYOF and SERPINB8) were all found to exhibit reduced protein expression in plasma samples from patients with cirrhosis. Similarly, the one gene that was shown to be down regulated in senescent hepatocytes (G6PC) was found to exhibit decreased protein expression in patients with cirrhosis.

For the twelve candidate genes, three of the ELISAs revealed significant differences in protein expression between fibrotic and cirrhotic samples (SPARC, CYR61, CSF1), while seven ELISAs revealed significant differences in protein expression between fibrotic and healthy samples (MVP, PGM2L1, ANXA3, MYOF, SERPINB8, G6PC and CSF1).

### Discussion

In conclusion, this study identified twelve candidate genes whose protein expression clearly distinguishes between patients with liver fibrosis and healthy controls.

Of these, CYR61, CSF1, IL-18, MRGPRX4 and LYVE1 exhibit increased protein expression in patients with liver fibrosis, and will therefore be useful biomarkers for detecting liver fibrosis. Whereas, SPARC, MVP, PGM2L11, ANXA3, MYOF, SERPINB8 and G6PC exhibit reduced protein expression in patients with liver fibrosis, and will therefore be useful biomarkers for excluding liver fibrosis.

Example 2 demonstrated selective insulin resistance in senescent hepatocytes. This finding is thought to explain the development of insulin resistance in advanced liver disease and suggests that hepatocyte senescence, progressive fibrosis and insulin resistance complicating chronic liver disease are inter-related.

Accordingly, protein expression of the twelve candidate genes identified in this study is also expected to distinguish between healthy individuals and those with diabetes mellitus. More particularly, it is believed that CYR61, CSF1, IL-18, MRGPRX4 and LYVE1 will exhibit increased protein expression in patients with diabetes mellitus, and will therefore be useful biomarkers for detecting diabetes mellitus. Whereas, it is believed that SPARC, MVP, PGM2L11, ANXA3, MYOF, SERPINB8 and G6PC will exhibit reduced protein expression in patients with diabetes mellitus, and will therefore be useful biomarkers for excluding diabetes mellitus.

What is claimed is:

1. (canceled)

2. A method for diagnosing, prognosing or evaluating the risk of liver disease in a patient, comprising: (a) determining an effective amount of one or more biomarkers in a sample obtained from a patient, wherein the one or more biomarkers are selected from the group consisting of G6PC, MVP, PGM2L1, ANXA3, SERPINB8, MYOF and SPARC; and (b) comparing the effective amount to a reference value, wherein a decrease in the effective amount relative to the reference value indicates that the patient has or may be at risk of developing liver disease.

3. (canceled)

4. The method of claim 1, wherein:
a. the one or more biomarkers are protein biomarkers;
b. the one or more biomarkers are nucleic acid biomarkers; or
c. the one or biomarkers are both protein and nucleic acid biomarkers.

5. The method of claim 1, wherein the sample is selected from the group consisting of blood, saliva, tears, sputum, urine, cerebral spinal fluid, cells, a cellular extract, a tissue specimen, a tissue biopsy, a stool specimen, or any combination thereof.

6. The method of claim 5, wherein the sample is blood.

7. The method of claim 6, wherein the sample is blood plasma.

8. (canceled)

9. A method for monitoring liver fibrosis, wherein said method comprises: (i) determining an effective amount of one or more biomarkers in a first sample obtained from a patient at a first time period; (ii) determining an effective amount of the one or more biomarkers in a sample obtained from the patient at one or more later time periods; and (iii) comparing the effective amount determined in step (ii) to the effective amount detected in step (i) to determine a difference in the effective amount of the one or more biomarkers, wherein the one or more biomarkers are selected from the group consisting of G6PC, MVP, PGM2L 1, ANXA3, SERPINB8, MYOF and SPARC; and wherein a decrease in the effective amount indicates progression or development of liver fibrosis.

10. The method of claim 9, wherein:
a. the one or more biomarkers are protein biomarkers;
b. the one or more biomarkers are nucleic acid biomarkers; or
c. the one or biomarkers are both protein and nucleic acid biomarkers.

11. The method of claim 9, wherein the sample is selected from the group consisting of blood, saliva, tears, sputum, urine, cerebral spinal fluid, cells, a cellular extract, a tissue specimen, a tissue biopsy, a stool specimen, or any combination thereof.

12. The method of claim 11, wherein the sample is blood.

13. The method of claim 12, wherein the sample is blood plasma.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A kit for carrying out the method of claim 1, wherein the kit comprises one or more reagents for measuring the one or more biomarkers.

29. The kit of claim 28, wherein the one or more reagents comprise nucleic acid sequences complementary to a portion of the nucleic acid biomarkers.

30. The kit of claim 28, wherein the one or more reagents comprise a plurality of antibodies that specifically bind one or more of the protein biomarkers of the invention.
